(19) United States
(12) Patent Application Publication
(10) Pub. No.: US 2025/0084476 A1
(43) Pub. Date: Mar. 13, 2025

(54) MULTIPLEXED IN SITU SIGNAL AMPLIFICATION WITH SPLINT LIGATION EXTENSION (SLX) PROBES

(71) Applicant: University of Washington, Seattle, WA (US)

(72) Inventors: Brian Beliveau, Seattle, WA (US); Conor Camplisson, Seattle, WA (US)

(73) Assignee: University of Washington, Seattle, WA (US)

(21) Appl. No.: 18/292,665

(22) PCT Filed: Jul. 28, 2022
(86) PCT No.: PCT/US2022/074268

§ 371 Date: Jan. 26, 2024

Related U.S. Application Data
(60) Provisional application No. 63/226,645, filed on Jul. 28, 2021.

(51) Int. Cl. C12Q 1/6876; C12Q 1/6841

(52) U.S. Cl. CPC C12Q 1/6876; C12Q 1/6841; C12Q 2600/16

(57) ABSTRACT

Embodiments of the present disclosure provide a single stranded nucleic acid construct with a predetermined number of binding sites, wherein the construct comprises a targeting domain, at least one signaling domain, and at least one splint oligonucleotide that hybridizes to complementary regions on the targeting domain and the signaling domain to form a ligation junction to link each domain to form a functional unit. The present disclosure also provides for methods of using the construct to quantify a target nucleic acid through signal intensity. In other embodiments, the disclosure provides use of the construct in multiplexed target detection methods.

### CROSS-REFERENCE(S) TO RELATED APPLICATION(S)

This application claims the benefit of U.S. Provisional Application No. 63/226,645, filed on Jul. 28, 2021.

### STATEMENT OF GOVERNMENT LICENSE RIGHTS

This invention was made with government support under Grant No. R35 GM137916, awarded by the National Institutes of Health. The government has certain rights in the invention.

### BACKGROUND

Fluorescence in situ hybridization (FISH) and related methods enable the spatial localization and quantification of nucleic acids in fixed cells and tissues. These methods have broad applications in both research (e.g., analyzing three-dimensional genome organization and spatial transcriptomics) and clinical diagnostics (e.g., diagnosing chromosomal abnormalities). Recent advances in FISH-related methods have enabled the multiplexed detection of large numbers of targets with relatively few available spectrally resolvable fluorescent channels by utilizing serial rounds of imaging, combinatorial labeling, ratiometric labeling, and combinations thereof.

Although the molecular strategies employed by these methods are wide ranging and diverse, a common thread is the need to amplify the intensity of fluorescent signals (e.g., FISH signals detected using quantitative microscopy) in order to produce sufficient signal to overcome any background auto-fluorescence: more intense fluorescent signals can also decrease imaging time, which increases the throughput of these methods.

For FISH-based technologies, signal amplification usually involves introducing additional ssDNA binding sites that localize fluorescent ‘imager’ strands for detection. There are enzymatic, hybridization-based, and chemical strategies used to facilitate the sufficient localization of fluorescent imager strands to create an amplified signal that can be detected robustly at the site of the target molecule.

There are a number of tradeoffs in several dimensions between extant amplification strategies. Most of these amplification strategies involve stochastic reaction dynamics that produce non-uniform populations of amplification products; for example, rolling circle amplification (RCA) produces a population of amplicons wherein the number of added binding sites for fluorescent imager strands is both non-uniform and not precisely known. While the amplified signal is sufficient for detection, this non-determinism introduces variation in fluorescence intensity that is unrelated to target molecule abundance and, thus, complicates quantification. Moreover, this artificially introduces a source of variation between samples and across experiments. An attractive property of the primer exchange reaction (PER) used in the signal amplification by exchange reaction (SABER) method is that PER is performed in vitro as a preparative step, allowing the same material to be used across several samples, eliminating amplification variability across samples. However, the reaction itself is still stochastic so the number of binding sites present in PER products is both non-uniform and unknown. Additionally, the catalytic nature of the reaction imposes sequence constraints, so it is generally not feasible to introduce arbitrary sequences of interest into PER products.

Accordingly, a need remains for an easy, flexible, and tunable signal amplification platform that is compatible with existing imaging system and avoids signal distortion resulting from stochastic production methods. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with the foregoing, in one aspect of the invention, the disclosure provides a single stranded nucleic acid construct with a predetermined number of binding sites, the construct comprising: a targeting domain, comprising at least one programmable unit with each unit comprising from a 5′ end to a 3′ end, a first orthogonal ligation adapter sequence, a target payload sequence comprising a predetermined number of binding sites, and a second orthogonal ligation adapter sequence: a signaling domain, comprising at least two programmable units with each unit comprising from a 5′ end to a 3′ end, a first orthogonal ligation adapter sequence, a signaling payload sequence comprising a predetermined number of binding sites, and a second orthogonal ligation adapter sequence; and at least one splint oligonucleotide that hybridizes to a complementary orthogonal ligation adapter sequence on a first unit and hybridizes to a complementary orthogonal ligation adapter sequence on a second unit to form a ligation junction to link the first programmable unit with the second programmable unit to form a functional unit, wherein the construct can have two or more functional units comprising the targeting domain of interest and at least one signaling domain of interest, and wherein the construct is amplified to produce the final construct product.

In another aspect of the invention, the disclosure provides a method of quantifying a target nucleic acid, the method comprising; (a) generating a first nucleic acid construct as recited above for targeting a nucleic acid of interest, wherein the construct comprises a predetermined number of binding sites for an imaging oligonucleotide; (b) generating a second nucleic acid construct for targeting a reference nucleic acid population, wherein the second construct comprises the same number of binding sites for the imaging oligonucleotide as the first construct generated in step (a); (c) contacting the target nucleic acid of interest with the first construct, wherein the first construct hybridizes to the nucleic acid of interest; (d) contacting the reference nucleic acid population with the second construct, wherein the second construct hybridizes to the reference nucleic acid population; (e) adding the imaging oligonucleotide to saturate the binding sites on the first construct and the second construct; (f) measuring the signal intensity from the imaging oligonucleotide bound to the first construct and the second construct; and (g) comparing the signal intensity from the imaging oligonucleotide bound to the first construct to the signal intensity from the imaging oligonucleotide bound to the second construct, wherein a lower signal intensity indicates the target nucleic acid is present at a lower level compared to the reference nucleic acid population, and wherein a higher signal intensity indicates the target nucleic acid is present at a higher level compared to the reference nucleic acid population.

In another aspect of the invention, the disclosure provides a multiplexed target detection method, the method comprising, (a) combining a plurality of single stranded nucleic acid constructs each with a predetermined number of binding sites for imaging oligonucleotides with a plurality of splint oligonucleotides, wherein the constructs each comprise; (i) a targeting domain, comprising at least one programmable unit with each unit comprising from a 5′ end to a 3′ end, a first orthogonal ligation adapter sequence, a target payload sequence comprising a predetermined number of binding sites, and a second orthogonal ligation adapter sequence; and (ii) a signaling domain, comprising at least two programmable units with each unit comprising from a 5′ end to a 3′ end, a first orthogonal ligation adapter sequence, a signaling payload sequence comprising a predetermined number of binding sites, and a second orthogonal ligation adapter sequence; and wherein the at least one splint oligonucleotide hybridizes to a complementary orthogonal ligation adapter sequence on a first unit and hybridizes to a complementary orthogonal ligation adapter sequence on a second unit to form a ligation junction to link the first programmable unit with the second programmable unit to form a first reaction mixture comprising a plurality of functional units; (b) combining the first reaction mixture produced in step (a) with a sample containing a plurality of nucleic acid targets, wherein the functional units in the first reaction mixture produced in step (a) each hybridize with a nucleic acid target through the targeting domain producing a second reaction mixture; (c) combining the second reaction mixture produced in step (b) with a plurality of imaging oligonucleotides, wherein the functional units in the second reaction mixture produced in step (b) each hybridize with an imaging oligonucleotide through the signaling domain; and (d) imaging the labeled functional units.

In another aspect of the invention, the disclosure provides a method of assembling a single stranded nucleic acid probe construct, comprising: providing reaction mixture comprising: a targeting oligonucleotide, comprising in order a first orthogonal ligation adapter sequence at a first end, a targeting payload sequence that hybridizes to a target sequence in a nucleic acid of interest, and a second orthogonal ligation adapter sequence at a second end, one or more signal oligonucleotides, each comprising in order a first orthogonal ligation adapter sequence at a first end, a signaling payload sequence that hybridizes to an imaging oligonucleotide, and a second orthogonal ligation adapter sequence at a second end, a first splint oligonucleotide with a first domain that hybridizes to the second orthogonal ligation adapter sequence of the targeting oligonucleotide and a second domain that hybridizes to the first orthogonal ligation adapter sequence of a first signal oligonucleotide: permitting the first splint oligonucleotide to hybridize to the second orthogonal ligation adapter sequence of the targeting oligonucleotide and the first orthogonal ligation adapter sequence of the first signal oligonucleotide, thereby bringing the second end of the targeting oligonucleotide and the first end of the first signal oligonucleotide into close proximity: ligating the second end of the targeting oligonucleotide to the first end of the first signal oligonucleotide to provide a ligated probe precursor.

In another aspect of the invention, the disclosure provides a method of tagging a target nucleic acid of interest with a detectable signal, the method comprising: contacting the target nucleic acid of interest with a single stranded nucleic acid construct as described above: contacting the single stranded nucleic acid construct with one or more imaging oligonucleotides; and providing conditions to allow the targeting payload sequence to hybridize to the target nucleic acid of interest and to allow the one or more imaging oligonucleotides to hybridize to one or more signaling payload sequences.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A through IC. Overview of Splint Ligation Extension (SLX) Probe Reagents. FIG. 1A. Principle of splint ligation: a complex is formed when two oligonucleotides each hybridize to a complementary “splint” oligonucleotide, forming a ligation junction, which is ligated to form a new longer product. FIG. 1B. Schematic of building block oligonucleotides (SLX Blocks), which contain functional “payload” sequences (e.g., primer binding sites, target homology sequences, and binding sites for fluorescent imager oligos) flanked by either one (i.e., for terminal blocks) or two (i.e., for internal blocks) orthogonal ligation adapter sequences. FIG. 1C. Synthesis and amplification of SLX probes. SLX blocks and complementary splint oligos hybridize to form a multi-molecular SLX ligation complex, which is then ligated and the ligation product is amplified to yield the final ssDNA probe reagents.

FIGS. 2A through 2B. In vitro validation of SLX probe synthesis products. FIG. 2A. Precisely programming the length of synthesized SLX probes (i.e., by including particular SLX blocks and corresponding splint oligos) modulates the number of binding sites available for fluorescent imager oligos in order to tune fluorescence intensity during FISH. FIG. 2B. Multiplexed one-pot synthesis of pools of SLX probes, each containing different targeting payload blocks that allow different SLX probes to be synthesized in parallel in a single ligation reaction.

### DETAILED DESCRIPTION

Fluorescence in situ hybridization (FISH) enables the interrogation of the location and abundance of target nucleic acid sequences in fixed cells and tissues. One common feature of the diverse existing FISH-based technologies is the amplification of fluorescent signal intensity through the introduction of additional binding sites for fluorescent ‘imager’ oligonucleotides. These additional binding sites are introduced through one of many enzymatic or chemical strategies. However, extant techniques to produce signal amplification rely on stochastic mechanisms to introduce binding sites, thus leading to signal amplification that is uncontrolled.

The inventors recognized that existing in situ detection technologies would benefit from a modular and scalable probe technology that provides precise control over the number of imager strand binding sites added during amplification, is free of sequence constraints, and is amenable to enhanced multiplexing strategies such as ratiometric and combinatorial labeling. Accordingly, as described in more detail below, the inventors developed a system of splint ligation extension (SLX) probes, which serve as a versatile and programmable ssDNA probe platform with several desirable properties for use with quantitative FISH-based methods. The utility and versatility of the programmable SLX probe synthesis and single- and multiplexed DNA-FISH were demonstrated on Human metaphase spreads using SLX probes against chromosomal targets.

### Composition for Detecting Nucleic Acids

In one aspect, the disclosure provides a single stranded nucleic acid construct with a predetermined number of binding sites. In some embodiments, the construct can comprise a targeting domain, a signaling domain, and at least one splint oligonucleotide.

As used herein a “targeting domain” comprises elements that are capable of hybridizing to a target nucleic acid of interest. The targeting domain can comprise at least one programmable unit with each unit comprising from a 5′ end to a 3′ end, a first orthogonal ligation adapter sequence, a target payload sequence, and a second orthogonal ligation adapter sequence. The target payload sequence can hybridize to a target nucleic acid of interest. In some embodiments, the target payload sequence can hybridize directly to the target nucleic acid of interest. In other embodiments, the target payload sequence can hybridize indirectly to the target nucleic acid of interest. For example, the target payload sequence can hybridize to a second nucleic acid and the second nucleic acid hybridizes to the target nucleic acid of interest. The target payload sequence includes at least one nucleotide sequence that is complementary to the nucleotide sequence of the target nucleic acid of interest, if hybridizing directly to the target nucleic acid of interest. In other embodiments, the target payload sequence includes at least one nucleotide sequence that is complementary to the second nucleotide sequence, if hybridizing indirectly to the target nucleic acid of interest.

In some embodiments, the targeting domain through its single target payload sequence can hybridize with one target nucleic acid of interest. In other embodiments, the targeting domain through two or more target payload sequences can hybridize with two or more target nucleic acids of interest. In some embodiments, a single target payload sequence can comprise a first nucleotide sequence complementary to a first target nucleic acid of interest and a second nucleotide sequence complementary to a second target nucleic acid of interest. In other embodiments, the single stranded nucleic acid construct can comprise two or more targeting domains and each targeting domain can comprise a target payload sequence, wherein each target payload sequence is specific to a target nucleic acid of interest.

In some embodiments, the target payload sequence can be any sequence that can be synthesized commercially as a single stranded DNA (ssDNA). In some embodiments, the target payload sequence can be up to 300 nucleotides in length. In some embodiments, the target payload sequence can be 1 to 300 nucleotides in length. In other embodiments, the target payload sequence can be at least 5 nucleotides in length: at least 10 nucleotides in length: at least 20 nucleotides in length: at least 30 nucleotides in length: at least 40 nucleotides in length: at least 50 nucleotides in length: at least 60 nucleotides in length: at least 70 nucleotides in length: at least 80 nucleotides in length: at least 90 nucleotides in length: at least 100 nucleotides in length: at least 125 nucleotides in length: at least 150 nucleotides in length: at least 175 nucleotides in length: at least 200 nucleotides in length: at least 225 nucleotides in length: at least 250 nucleotides in length; at least 275 nucleotides in length: or 300 nucleotides in length.

A target nucleic acid of interest can be essentially any nucleic acid that is desirably detected in a sample. In some embodiments, a target nucleic acid of interest can be a DNA, a chromosomal DNA, an RNA (e.g., a cytoplasmic RNA), an mRNA, a microRNA, a ribosomal RNA. In other embodiments, the target nucleic acid of interest can be a nucleic acid endogenous to a cell. In still other embodiments, the target nucleic acid of interest can be a nucleic acid introduced to or expressed in the cell by infection of the cell with a pathogen, for example, a viral or bacterial genomic RNA or DNA, a plasmid, a viral or bacterial mRNA.

In some embodiments, the targeting domain comprises one or more functional sequences encoding one or more functional linkers, adapters, barcode tags, or other functional domains, in any order or combination, wherein the one or more sequences are disposed in a location between the first orthogonal ligation adapter sequence and the second orthogonal ligation adapter sequence.

As used herein, an “orthogonal ligation adapter sequence” enable the desired ligation product while preventing unwanted side products resulting from cross-hybridization or non-specific ligation. A set of orthogonal adapter sequences are computationally designed with favorable thermodynamic properties for specific complex formation via hybridization and ligation at 25° C. In some embodiments, the orthogonal adapter sequence is complementary to at least one splint oligonucleotide, enabling the program of a ligation junction between a particular targeting domain via its orthogonal adapter sequences hybridizing with a corresponding splint oligonucleotide.

As used herein, a “signaling domain” comprises elements that enable binding of at least signal generating element. The signaling domain can comprise at least two programmable units with each unit comprising from a 5′ end to a 3′ end, a first orthogonal ligation adapter sequence, a signaling payload sequence comprising a predetermined number of binding sites, and a second orthogonal ligation adapter sequence. In some embodiments, the signaling payload sequence can bind (e.g., hybridize) to any signal generating element well-known to one of ordinary skill in the art. In other embodiments, the signaling payload sequence can bind to any molecule, directly or indirectly (e.g., biotin/avidin), that can allow the target to be detected. In some embodiments, the molecule comprises a nucleic acid binding portion such as an oligonucleotide and an enzyme that binds to the signaling payload sequence, wherein the binding of the enzyme to the signaling payload sequence can be covalent or through a high affinity binding interaction such as biotin/avidin or other similar high affinity binding molecules. In still other embodiments, the signal generating element can include an imaging oligonucleotide. In other embodiments, the signal generating element can include any labeling method for direct visualization, which can include, but is not limited to a fluorescent label or a chromogenic label. In other embodiments, the signal generating element can include direct addition of detectable metal isotopes, or an enzymatic or chemical reaction that generates a fluorescent, chromogenic, or other detectable signal, as understood by one skilled in the art.

In some embodiments, the construct comprises a plurality of signal domains, wherein a second and optionally a third, fourth, or fifth, signal domain comprises the signaling payload sequence, wherein the respective signaling payload sequence hybridizes to a same or different imaging oligonucleotide of interest, in any combination or order. For example, in some embodiments, the signaling payload sequence can comprise a predetermined number of binding sites for the labeling molecule (e.g., imaging oligonucleotide). In some embodiments, the number of binding sites can be calibrated for a specific signal intensity as determined by one skilled in the art. For example, the number of binding sites for the labeling molecule can be at least 1, at least 2, at least 3, at least 4, at least 5, at least 6, at least 7, at least 8, at least 9, at least 10 or more. In some embodiments, the single stranded nucleic acid construct can comprise 2 or more signaling domains each comprising a signaling payload sequence with at least 1, at least 2, at least 3, at least 4, at least 5, at least 6, at least 7, at least 8, at least 9, at least 10 or more binding sites for a labeling molecule. In some embodiments, the signaling payload sequences can each have the same number of binding sites to bind the same number of labeling molecules. In other embodiments, the signaling payload sequence can each have a different number of binding sites to bind a different number of labeling molecules. In some embodiments, the binding sites are identical. In other embodiment, the binding sites are different.

In some embodiments, the signaling payload comprises a sequence between 5-50 nucleotides in length. In some embodiments, the signaling payload comprises a sequence 5 nucleotides in length. In some embodiments, the signaling payload comprises a sequence 10 nucleotides in length. In other embodiments, the signaling payload comprises a sequence 15 nucleotides in length. In other embodiments, the signaling payload comprises a sequence 20 nucleotides in length. In other embodiments, the signaling payload comprises a sequence 25 nucleotides in length. In other embodiments, the signaling payload comprises a sequence 30 nucleotides in length. In other embodiments, the signaling payload comprises a sequence 35 nucleotides in length. In other embodiments, the signaling payload comprises a sequence 40 nucleotides in length. In other embodiments, the signaling payload comprises a sequence 45 nucleotides in length. In other embodiments, the signaling payload comprises a sequence 50 nucleotides in length.

In other embodiments, the signaling payload sequence can comprise a predetermined number of binding sites for any functional sequence for delivering cargo to a target nucleic acid of interest. In some embodiments, the signaling payload sequence can include binding sites for cargo that can include, but is not limited to, linkers, adapters, barcodes, and the like.

As used herein, a “splint oligonucleotide” functions to connect the “blocks” comprising at least one targeting domain and/or at least one signaling domain to form at least one functional unit comprising a plurality of targeting domains and/or signaling domains. The at least one splint oligonucleotide hybridizes to a complementary orthogonal ligation adapter sequence on a first unit and hybridizes to a complementary orthogonal ligation adapter sequence on a second unit to form a ligation junction to link the first programmable unit with the second programmable unit to form a functional unit. In some embodiments, splint oligonucleotides can be used to create a ligation junction between two or more, three or more, four or more, five or more, six or more, seven or more, eight or more, nine or more, or 10 or more oligonucleotide termini (e.g., block comprising at least one targeting domain and/or at least one signaling domain).

In some embodiments, the first orthogonal ligation adapter sequences of the targeting domain and the one or more signal domains can be different. In other embodiments, the second orthogonal ligation adapter sequences of the targeting domain and the one or more signal domains can be different. In other embodiments, the first orthogonal ligation adapter sequence and the second orthogonal ligation adapter sequence in any single domain can be different.

As such, multiplexed splint ligation of oligonucleotide blocks containing targeting domains and/or signaling domains (e.g., target payload sequences and signaling payload sequence) to produce arbitrary, modular single stranded nucleic acid constructs from short oligonucleotides and the product (e.g., modular single stranded nucleic acid constructs) can be produced at scale using any ssDNA amplification strategies well-known in the art. In some embodiments, terminal PCR adapter sequences are added during ligation and the full-length ligation product is selectively amplified using terminal PCR primers. In some embodiments, a T7 promoter added during PCR renders the PCR product a valid template for T7 in vitro transcription (IVT), producing an RNA version of the SLX probes, which is reverse-transcribed to yield the final ssDNA SLX probes at scale. In other embodiments, other well-known methods can be used to amplify the ligation products and convert them back to ssDNA constructs. For example, in some embodiments, PCR can be followed by selective exonuclease digestion. In other embodiments, PCR can be followed by DNA nicking and gel purification. In still other embodiments circle-to-circle DNA amplification can be performed.

In some embodiments, the single stranded nucleic acid construct can comprise a terminal domain, comprising a first orthogonal ligation adapter sequence and a primer payload sequence that comprises a primer binding site. In some embodiments, the first terminal domain is covalently joined to the orthogonal ligation adapter sequence at the 5′ end of the construct and a second terminal domain is covalently joined to the orthogonal ligation adapter sequence at the 3′ end of the construct, wherein the terminal domain allows for amplification of the construct.

In other embodiments, the construct further comprises a modified 5′ end. In still other embodiments, the construct further comprises a modified 3′ end. In other embodiments, the construct further comprises a modified 5′ end and a modified 3′ end. The modified 5′ and/or 3′ termini protect the construct from exonuclease digestion. Methods to modify the 5′ and/or 3′ termini from exonuclease digestion can be any method well-known in the art, including but not limited to phosphate modification, and the introduction of phosphorothioate bonds between the last 3-5 nucleotides at the 5′ and/or 3′ ends of the oligonucleotide.

### Methods of Quantifying Nucleic Acid Abundance

In another aspect, the disclosure provides a method of quantifying a target nucleic acid. In some embodiments, the method can comprise; (a) generating a first nucleic acid construct as described above for targeting a nucleic acid of interest, wherein the construct comprises a predetermined number of binding sites for an imaging oligonucleotide; (b) generating a second nucleic acid construct for targeting a reference nucleic acid population, wherein the second construct comprises the same number of binding sites for the imaging oligonucleotide as the first construct generated in step (a); (c) contacting the target nucleic acid of interest with the first construct, wherein the first construct hybridizes to the nucleic acid of interest; (d) contacting the reference nucleic acid population with the second construct, wherein the second construct hybridizes to the reference nucleic acid population; (e) adding the imaging oligonucleotide to saturate the binding sites on the first construct and the second construct; (f) measuring the signal intensity from the imaging oligonucleotide bound to the first construct and the second construct; and (g) comparing the signal intensity from the imaging oligonucleotide bound to the first construct to the signal intensity from the imaging oligonucleotide bound to the second construct, wherein a lower signal intensity indicates the target nucleic acid is present at a lower level compared to the reference nucleic acid population, and wherein a higher signal intensity indicates the target nucleic acid is present at a higher level compared to the reference nucleic acid population.

As used herein, a “reference nucleic acid population” is a nucleic acid population with a known abundance that can be used as a control for comparison to the target nucleic acid of interest. The reference nucleic acid population can be any nucleic acid population with a known abundance as determined by one skilled in the art. Additionally, as used herein, a second nucleic acid construct for targeting a reference nucleic acid population can be generated as described above. In some embodiments, the second nucleic acid construct can be used as a control construct to compare signal intensity generated by the target nucleic acid of interest via the first construct. In some embodiments, the second nucleic acid construct has the same number of binding sites (e.g., to bind the imaging oligonucleotide) as the first construct. As described throughout this disclosure, the single stranded construct can be calibrated to have a predetermined number (i.e., programmed number) of binding sites and having a predetermined number of binding sites allows for single intensity to be used to encode information, for example, nucleic acid abundance.

In other embodiments, signal intensity can be combined with a second or third information channel (e.g., color) to increase the number of targeted nucleic acids of interest that can be imaged simultaneously. In other embodiments, a second (or additional) nucleic acid constructs can be used to target multiple nucleic acids of interest. Each construct can have multiple channels (e.g., color) for encoding information, which is then compared to calibration standards or internal normalization to quantitate the target nucleic acid of interest.

In some embodiments, quantifying nucleic acids of interest in biological samples can be broadly categorized into two types of methods: “whole-sample” and “in situ” detection. In the whole-sample detection method, the cells in the sample are lysed, which releases the molecules contained in the cells, including the nucleic acid analytes, into sample solution. Then the quantities of the nucleic acid analytes of the entire biological sample are measured in the solution. In the in situ detection method, the nucleic acid analytes are fixed within the host cells and their quantities are measured at an individual cell level.

In another aspect, the disclosure provides a multiplexed target detection method. the method can comprise (a) combining a plurality of single stranded nucleic acid constructs each with a predetermined number of binding sites for imaging oligonucleotides with a plurality of splint oligonucleotides, wherein the constructs each comprise; (i) a targeting domain, comprising at least one programmable unit with each unit comprising from a 5′ end to a 3′ end, a first orthogonal ligation adapter sequence, a target payload sequence comprising a predetermined number of binding sites, and a second orthogonal ligation adapter sequence; and (ii) a signaling domain, comprising at least two programmable units with each unit comprising from a 5′ end to a 3′ end, a first orthogonal ligation adapter sequence, a signaling payload sequence comprising a predetermined number of binding sites, and a second orthogonal ligation adapter sequence; and wherein the at least one splint oligonucleotide hybridizes to a complementary orthogonal ligation adapter sequence on a first unit and hybridizes to a complementary orthogonal ligation adapter sequence on a second unit to form a ligation junction to link the first programmable unit with the second programmable unit to form a first reaction mixture comprising a plurality of functional units; (b) combining the first reaction mixture produced in step (a) with a sample containing a plurality of nucleic acid targets, wherein the functional units in the first reaction mixture produced in step (a) each hybridize with a nucleic acid target through the targeting domain producing a second reaction mixture; (c) combining the second reaction mixture produced in step (b) with a plurality of imaging oligonucleotides, wherein the functional units in the second reaction mixture produced in step (b) each hybridize with an imaging oligonucleotide through the signaling domain; and (d) imaging the labeled functional units. As used herein, imaging the labeled functional units refers to quantifying the signal from the imaging oligonucleotide. Methods to quantify a signal from an imaging oligonucleotide can comprise any method well-known in the art.

In some embodiments, the method can be used to simultaneously quantify more than two nucleic acid targets of interest (e.g., three nucleic acid targets of interest). As such, the sample comprises at least three nucleic acid targets, and the methods include: providing at least three constructs each comprising a targeting domain each comprising a target payload sequence that can hybridize to the first, second, and third, respectively, nucleic acids of interest. The constructs would each additional comprise a signaling domain each comprising a signaling payload sequence that can hybridize to a first, second, and third, respectively, imaging oligonucleotides, wherein the first, second, and third imaging oligonucleotides can each be distinguishable. In other embodiments, fourth, fifth, sixth, or more nucleic acid targets of interest are similarly simultaneously detected in the cell if desired. In some embodiments, the construct would comprise a fourth, fifth, sixth, or more targeting domains that each hybridize to a fourth, fifth, sixth, or more nucleic acid targets of interest, and a fourth, fifth, sixth, or more signaling domains that each hybridize to a fourth, fifth, sixth, or more imaging oligonucleotides that can each be distinguishable.

In another aspect, the disclosure provides a method of assembling a single stranded nucleic acid probe construct. The method can comprise providing reaction mixture comprising a targeting oligonucleotide, comprising in order a first orthogonal ligation adapter sequence at a first end, a targeting payload sequence that hybridizes to a target sequence in a nucleic acid of interest, and a second orthogonal ligation adapter sequence at a second end, one or more signal oligonucleotides, each comprising in order a first orthogonal ligation adapter sequence at a first end, a signaling payload sequence that hybridizes to an imaging oligonucleotide, and a second orthogonal ligation adapter sequence at a second end, a first splint oligonucleotide with a first domain that hybridizes to the second orthogonal ligation adapter sequence of the targeting oligonucleotide and a second domain that hybridizes to the first orthogonal ligation adapter sequence of a first signal oligonucleotide: permitting the first splint oligonucleotide to hybridize to the second orthogonal ligation adapter sequence of the targeting oligonucleotide and the first orthogonal ligation adapter sequence of the first signal oligonucleotide, thereby bringing the second end of the targeting oligonucleotide and the first end of the first signal oligo into close proximity: ligating the second end of the targeting oligonucleotide to the first end of the first signal oligonucleotide to provide a ligated probe precursor.

In some embodiments, the method comprises attaching end adapters to each end of the ligated probe precursor, wherein each end adapter comprises a primer binding site.

In other embodiments, the reaction mixture further comprises: a first terminal oligonucleotide, comprising a first orthogonal ligation adapter sequence at a first end and a primer payload sequence that comprises a first primer binding site, a second terminal oligonucleotide, comprising a first orthogonal ligation adapter sequence at a first end and a primer payload sequence that comprises a second primer binding site, wherein the first primer binding site and the second primer binding site are the same or different, a first end splint oligonucleotide with a first domain that hybridizes to the first orthogonal ligation adapter sequence of the first terminal oligonucleotide and a second domain that hybridizes to the first orthogonal ligation adapter sequence of the targeting oligonucleotide, and a second end splint oligonucleotide with a first domain that hybridizes to the first orthogonal ligation adapter sequence of the second terminal oligonucleotide and a second domain that hybridizes to the second orthogonal ligation adapter sequence of a signal oligonucleotide; and the hybridizing step further comprises: permitting the first end splint oligonucleotide to hybridize to the first orthogonal ligation adapter sequence of the first terminal oligonucleotide and the first orthogonal ligation adapter sequence of the targeting oligonucleotide, thereby bringing the first end of the first terminal oligonucleotide and the first end of the targeting oligonucleotide into close proximity; and permitting the second end splint oligonucleotide to hybridize to the first orthogonal ligation adapter sequence of the second terminal oligonucleotide and the second orthogonal ligation adapter sequence of a signal oligonucleotide, thereby bringing the first end of the second terminal oligo and the second end of the signal oligonucleotide into close proximity; and the ligating step further comprises: ligating the first end of the first terminal oligonucleotide to the first end of the targeting oligonucleotide; and ligating the first end of the second terminal oligonucleotide to the second end of the signal oligonucleotide.

In still other embodiments, the method further comprises amplifying the ligated probe precursor with primers that bind to the first primer biding site and the second primer binding site to provide an amplified probe precursor. In other embodiments, the method further comprises transcribing the amplified probe precursor to provide an RNA probe precursor. In other embodiments, the method further comprises reverse transcribing the RNA probe precursor to provide a single stranded DNA probe construct. In still other embodiments, the method further comprises subjecting the amplified probe precursor to exonuclease digestion to provide a single stranded DNA probe construct. In still other embodiments, the method further comprises subjecting the amplified probe precursor to DNA nicking and gel purification to provide a single stranded DNA probe construct. In still other embodiments, the method further comprises performing circle-to-circle DNA amplification to provide a single stranded DNA probe construct.

In some embodiments, the method comprises incorporating a plurality of signal oligonucleotides into the ligated probe construct, wherein each additional signal oligonucleotide is incorporated by: providing at least one additional splint oligonucleotide with a first domain that hybridizes to the second orthogonal ligation adapter sequence of an adjacent oligonucleotide and a second domain that hybridizes to the first orthogonal ligation adapter sequence of the additional signal oligonucleotide, permitting the additional splint oligonucleotide to hybridize to the second orthogonal ligation adapter sequence of the adjacent oligonucleotide and the first orthogonal ligation adapter sequence of an additional signal oligonucleotide, thereby bringing the second end of the adjacent oligonucleotide and the first end of the additional oligonucleotide into close proximity; and ligating the second end of the adjacent oligonucleotide to the first end of the additional oligonucleotide to provide a ligated probe precursor.

In some embodiments, the method comprising incorporating between 2 and 20 signal oligonucleotides. In other embodiments, each of the plurality of signal oligonucleotides comprise a signaling payload sequence, wherein the signaling payload sequence hybridizes to a same or different imaging oligonucleotide, in any combination or order.

In other embodiments, the first orthogonal ligation adapter sequence of the targeting oligonucleotide and the one or more signal oligonucleotide are different. In other embodiments, the second orthogonal ligation adapter sequences of the targeting oligonucleotide and the one or more signal oligonucleotides are different. In still other embodiments, the first orthogonal ligation adapter sequence and the second orthogonal ligation adapter sequence in any single domain are different.

In another aspect, the disclosure provides a single stranded nucleic acid probe construct produced by the methods recited above.

In another aspect, the disclosure provides a method of tagging a target nucleic acid of interest with a detectable signal. The method can comprise contacting the target nucleic acid of interest with a single stranded nucleic acid construct as described above; contacting the single stranded nucleic acid construct with one or more imaging oligonucleotides; and providing conditions to allow the targeting payload sequence to hybridize to the target nucleic acid of interest and to allow the one or more imaging oligonucleotides to hybridize to one or more signaling payload sequences. In some embodiments, the method comprises detecting the signal produced by one or more imager oligonucleotides. In some embodiments, the target nucleic acid of interest is a chromosomal DNA.

### Additional Definitions

As used herein, the terms “bind” and “attach” refer to both covalent interactions and noncovalent interactions. A covalent interaction is a chemical linkage between two atoms or radicals formed by the sharing of a pair of electrons (i.e., a single bond), two pairs of electrons (i.e., a double bond) or three pairs of electrons (i.e., a triple bond). Covalent interactions are also known in the art as electron pair interactions or electron pair bonds. Noncovalent interactions include, but are not limited to, van der Waals interactions, hydrogen bonds, weak chemical bonds (i.e., via short-range noncovalent forces), hydrophobic interactions, ionic bonds and the like. A review of noncovalent interactions can be found in Alberts et al., in Molecular Biology of the Cell. 3d edition, Garland Publishing, 1994.

The term “primer” as used herein refers to an oligonucleotide which is capable of annealing to the oligonucleotide template sequence allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The primer is preferably single stranded for maximum efficiency. Preferably, the primer is an oligodeoxyribonucleotide. The primer must be sufficiently long to prime the synthesis of extension products in the presence of the oligonucleotide template sequence. The exact lengths of the primers will depend on many factors, including temperature and composition (A/T en G/C content) of primer. A pair of bi-directional primers consists of one forward and one reverse primer as commonly used in the art of DNA amplification such as in PCR amplification. The primer may include a label.

It will be understood that “primer”, as used herein, may refer to more than one primer, particularly in the case where there is some ambiguity in the information regarding the terminal sequence(s) of the target region to be amplified. Hence, a “primer” includes a collection of primer oligonucleotides containing sequences representing the possible variations in the sequence or includes nucleotides which allow a typical base pairing.

The oligonucleotide primers may be prepared by any suitable method. Methods for preparing oligonucleotides of specific sequence are known in the art, and include, for example, cloning and restriction of appropriate sequences, and direct chemical synthesis. Chemical synthesis methods may include, for example, the phospho di- or tri-ester method, the diethylphosphoramidate method and the solid support method disclosed in e.g., U.S. Pat. No. 4,458,066. The primers may be labeled, if desired, by incorporating means detectable by for instance spectroscopic, fluorescence, photochemical, biochemical, immunochemical, or chemical means.

The terms “nucleic acid,” “nucleic acid molecule,” “nucleic acid sequence,” “nucleic acid fragment,” “oligonucleotide” and “polynucleotide” are used interchangeably and are intended to include, but not limited to, a polymeric form of nucleotides that may have various lengths, either deoxyribonucleotides or ribonucleotides, or analogs thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, ribozymes, small interfering RNA (siRNA), cDNA, recombinant polynucleotides, branched polynucleotides, plasmids, vectors, isolated DNA of a sequence, isolated RNA of a sequence, nucleic acid probes, and primers. Oligonucleotides or polynucleotides useful in the methods described herein may comprise natural nucleic acid sequences and variants thereof, artificial nucleic acid sequences, or a combination of such sequences. Oligonucleotides or polynucleotides may be single stranded or double stranded. Additionally, “nucleic acid” as used in the specification and claims of this patent application, when referring to a target molecule or target sequence, “nucleic acid” means RNA or DNA, including in either case oligonucleotides with natural nucleotides and phosphodiester bonds.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule: alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Oligonucleotide sequences, such as single stranded oligonucleotide sequences useful in the rolling circle amplification methods described herein, may be isolated from natural sources, synthesized or purchased from commercial sources. In certain exemplary embodiments, oligonucleotide sequences may be prepared using one or more of the phosphoramidite linkers and/or sequencing by ligation methods known to those of skill in the art. Oligonucleotide sequences may also be prepared by any suitable method, e.g., standard phosphoramidite methods such as those described herein below as well as those described by Beaucage and Carruthers ((1981)Tetrahedron Lett. 22:1859) or the triester method according to Matteucci et al. (1981) J. Am. Chem. Soc. 103:3185), or by other chemical methods using either a commercial automated oligonucleotide synthesizer or high-throughput, high-density array methods known in the art (see U.S. Pat. Nos. 5,602,244, 5,574,146, 5,554,744, 5,428,148, 5,264,566, 5,141,813, 5,959,463, 4,861,571 and 4,659,774, incorporated herein by reference in its entirety for all purposes). Pre-synthesized oligonucleotides, useful in the rolling circle amplification methods described herein, may also be obtained commercially from a variety of vendors.

“Adjacently”. As used in the specification and claims of this patent application to describe the hybridization two oligonucleotides and its complementary ‘splint’ oligonucleotide, “adjacently” means sites that are sufficiently close to each other to permit hybridization between two oligonucleotides and a complementary splint oligonucleotide.

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, New York (2001); Ausubel, F. M., et al. (eds.), Current Protocols in Molecular Biology, John Wiley & Sons, New York (2010); and Coligan, J. E., et al. (eds.), Current Protocols in Immunology, John Wiley & Sons, New York (2010) Mirzaei, H. and Carrasco, M. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed in this description and/or the claims are provided here. The definitions are provided to aid in describing particular embodiments and are not intended to limit the claimed subject matter, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, which is to indicate, in the sense of “including, but not limited to.” Words using the singular or plural number also include the plural and singular number, respectively. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

Embodiment 1. A single stranded nucleic acid construct with a predetermined number of binding sites, the construct comprising: a targeting domain, comprising at least one programmable unit with each unit comprising from a 5′ end to a 3′ end, a first orthogonal ligation adapter sequence, a target payload sequence comprising a predetermined number of binding sites, and a second orthogonal ligation adapter sequence: a signaling domain, comprising at least two programmable units with each unit comprising from a 5′ end to a 3′ end, a first orthogonal ligation adapter sequence, a signaling payload sequence comprising a predetermined number of binding sites, and a second orthogonal ligation adapter sequence; and at least one splint oligonucleotide that hybridizes to a complementary orthogonal ligation adapter sequence on a first unit and hybridizes to a complementary orthogonal ligation adapter sequence on a second unit to form a ligation junction to link the first programmable unit with the second programmable unit to form a functional unit, wherein the construct can have two or more functional units comprising the targeting domain of interest and at least one signaling domain of interest, and wherein the construct is amplified to produce the final construct product.

Embodiment 2. The construct of embodiment 1, wherein the targeting payload sequence hybridizes to a target sequence in a nucleic acid of interest.

Embodiment 3. The construct of embodiment 1, wherein the signaling payload sequence hybridizes to an imaging oligonucleotide of interest.

Embodiment 4. The construct of embodiment 1, wherein the targeting domain comprises one or more functional sequences encoding one or more functional linkers, adapters, barcode tags, or other functional domains, in any order or combination, wherein the one or more sequences are disposed in a location between the first orthogonal ligation adapter sequence and the second orthogonal ligation adapter sequence.

Embodiment 5. The construct as in any one of embodiments 1-4, further comprising: a terminal domain, comprising a first orthogonal ligation adapter sequence and a primer payload sequence that comprises a primer binding site, and wherein a first terminal domain is covalently joined to the orthogonal ligation adapter sequence at the 5′ end of the construct and a second terminal domain is covalently joined to the orthogonal ligation adapter sequence at the 3′ end of the construct, wherein the terminal domain allows for amplification of the construct.

Embodiment 6. The construct as in any one of embodiments 1-5, comprising a plurality of signal domains, wherein a second and optionally a third, fourth, or fifth, signal domain comprises the signaling payload sequence, wherein the respective signaling payload sequence hybridizes to a same or different imaging oligonucleotide of interest, in any combination or order.

Embodiment 7. The construct as in any one of embodiments 1-6, wherein the first orthogonal ligation adapter sequences of the targeting domain and the one or more signal domains are different.

Embodiment 8. The construct as in any one of embodiments 1-7, wherein the targeting payload comprises a sequence up to 300 nucleotides in length.

Embodiment 9. The construct as in any one of embodiments 1-8, wherein the signaling payload comprises a sequence between 10-30 nucleotides in length.

Embodiment 10. The construct as in any one of embodiments 1-9, wherein the signaling payload sequence comprises two or more binding sites.

Embodiment 11. The construct of embodiment 10, wherein the binding sites are identical.

Embodiment 12. The construct of embodiment 10, wherein each binding site is different.

Embodiment 13. The construct as in any one of embodiments 1-12, wherein the second orthogonal ligation adapter sequences of the targeting domain and the one or more signal domains are different.

Embodiment 14. The construct as in any one of embodiments 1-12, wherein the first orthogonal ligation adapter sequence and the second orthogonal ligation adapter sequence in any single domain are different.

Embodiment 15. The construct as in any one of embodiments 1-14, wherein the construct further comprises a modified 5′ end and a modified 3′ end, wherein the modified 5′ end and the modified 3′ end protect the construct from exonuclease digestion.

Embodiment 16. The construct as in any one of embodiments 1-15, wherein the nucleic acid of interest comprises DNA and/or RNA.

Embodiment 17. The construct as in any one of embodiments 1-15, wherein the imaging oligonucleotide of interest is a fluorophore.

Embodiment 18. A method of quantifying a target nucleic acid, the method comprising; (a) generating a first nucleic acid construct as recited in embodiment 1 for targeting a nucleic acid of interest, wherein the construct comprises a predetermined number of binding sites for an imaging oligonucleotide; (b) generating a second nucleic acid construct for targeting a reference nucleic acid population, wherein the second construct comprises the same number of binding sites for the imaging oligonucleotide as the first construct generated in step (a); (c) contacting the target nucleic acid of interest with the first construct, wherein the first construct hybridizes to the nucleic acid of interest; (d) contacting the reference nucleic acid population with the second construct, wherein the second construct hybridizes to the reference nucleic acid population; (e) adding the imaging oligonucleotide to saturate the binding sites on the first construct and the second construct; (f) measuring the signal intensity from the imaging oligonucleotide bound to the first construct and the second construct; and (g) comparing the signal intensity from the imaging oligonucleotide bound to the first construct to the signal intensity from the imaging oligonucleotide bound to the second construct, wherein a lower signal intensity indicates the target nucleic acid is present at a lower level compared to the reference nucleic acid population, and wherein a higher signal intensity indicates the target nucleic acid is present at a higher level compared to the reference nucleic acid population.

Embodiment 19. The method of embodiment 18, wherein the second nucleic acid construct comprises: a targeting domain, comprising at least one programmable unit with each unit comprising from a 5′ end to a 3′ end, a first orthogonal ligation adapter sequence, a target reference nucleic acid payload sequence comprising a predetermined number of binding sites, and a second orthogonal ligation adapter sequence: a signaling domain, comprising at least two programmable units with each unit comprising from a 5′ end to a 3′ end, a first orthogonal ligation adapter sequence, a signaling payload sequence comprising a predetermined number of binding sites that match the first construct, and a second orthogonal ligation adapter sequence; and at least one splint oligonucleotide that hybridizes to a complementary orthogonal ligation adapter sequence on a first unit and hybridizes to a complementary orthogonal ligation adapter sequence on a second unit to form a ligation junction to link the first programmable unit with the second programmable unit to form a functional unit, wherein the construct can have two or more functional units comprising the targeting domain of interest and at least one signaling domain of interest, and wherein the construct is amplified to produce the final construct product.

Embodiment 20. A multiplexed target detection method, the method comprising: (a) combining a plurality of single stranded nucleic acid constructs each with a predetermined number of binding sites for imaging oligonucleotides with a plurality of splint oligonucleotides, wherein the constructs each comprise; (i) a targeting domain, comprising at least one programmable unit with each unit comprising from a 5′ end to a 3′ end, a first orthogonal ligation adapter sequence, a target payload sequence comprising a predetermined number of binding sites, and a second orthogonal ligation adapter sequence; and (ii) a signaling domain, comprising at least two programmable units with each unit comprising from a 5′ end to a 3′ end, a first orthogonal ligation adapter sequence, a signaling payload sequence comprising a predetermined number of binding sites, and a second orthogonal ligation adapter sequence; and wherein the at least one splint oligonucleotide hybridizes to a complementary orthogonal ligation adapter sequence on a first unit and hybridizes to a complementary orthogonal ligation adapter sequence on a second unit to form a ligation junction to link the first programmable unit with the second programmable unit to form a first reaction mixture comprising a plurality of functional units; (b) combining the first reaction mixture produced in step (a) with a sample containing a plurality of nucleic acid targets, wherein the functional units in the first reaction mixture produced in step (a) each hybridize with a nucleic acid target through the targeting domain producing a second reaction mixture; (c) combining the second reaction mixture produced in step (b) with a plurality of imaging oligonucleotides, wherein the functional units in the second reaction mixture produced in step (b) each hybridize with an imaging oligonucleotide through the signaling domain; and (d) imaging the labeled functional units.

Embodiment 21. The method of embodiment 20, wherein the targeting payload sequence of the functional unit hybridizes to a sequence in the nucleic acid target.

Embodiment 22. The method of embodiment 20, wherein the signaling payload sequence hybridizes to the imaging oligonucleotide.

Embodiment 23. The method of embodiment 20, wherein the targeting domain comprises one or more functional sequences encoding one or more functional linkers, adapters, barcode tags, or other functional domains, in any order or combination, wherein the one or more sequences are disposed in a location between the first orthogonal ligation adapter sequence and the second orthogonal ligation adapter sequence.

Embodiment 24. The method as in any one of embodiments 20-23, further comprising: a terminal domain, comprising a first orthogonal ligation adapter sequence and a primer payload sequence that comprises a primer binding site, and wherein a first terminal domain is covalently joined to the orthogonal ligation adapter sequence at the 5′ end of the construct and a second terminal domain is covalently joined to the orthogonal ligation adapter sequence at the 3′ end of the construct, wherein the terminal domain allows for amplification of the construct.

Embodiment 25. The method as in any one of embodiments 20-24, comprising a plurality of signal domains, wherein a second and optionally third, fourth, or fifth, signal domain comprises the signaling payload sequence, wherein the signaling payload sequence hybridizes to a same or different imaging oligonucleotide, in any combination or order.

Embodiment 26. The method as in any one of embodiments 20-25, wherein the first orthogonal ligation adapter sequences of the targeting domain and the one or more signal domains are different.

Embodiment 27. The method as in any one of embodiments 20-26, wherein the targeting payload comprises a sequence up to 300 nucleotides in length.

Embodiment 28. The method as in any one of embodiments 20-26, wherein the signaling payload comprises a sequence between 10-30 nucleotides in length.

Embodiment 29. The method as in any one of embodiments 20-28, wherein the signaling payload sequence comprises two or more binding sites.

Embodiment 30. The method of embodiment 29, wherein the binding sites are identical.

Embodiment 31. The method of embodiment 29, wherein each binding site is different.

Embodiment 32. The method as in any one of embodiments 20-31, wherein the second orthogonal ligation adapter sequences of the targeting domain and the one or more signal domains are different.

Embodiment 33. The method as in any one of embodiments 20-31, wherein the first orthogonal ligation adapter sequence and the second orthogonal ligation adapter sequence in any single domain are different.

Embodiment 34. The method as in any one of embodiments 20-33, wherein the first construct and/or the second construct further comprise a modified 5′ end and a modified 3′ end, wherein the modified 5′ end and the modified 3′ end protect the first construct and/or the second construct from exonuclease digestion.

Embodiment 35. The method as in any one of embodiments 20-34, wherein the nucleic acid of interest comprises DNA and/or RNA.

Embodiment 36. The method as in any one of embodiments 20-35, wherein the imaging oligonucleotide of interest of a fluorophore.

Embodiment 37. A method of assembling a single stranded nucleic acid probe construct, comprising: providing reaction mixture comprising: a targeting oligonucleotide, comprising in order a first orthogonal ligation adapter sequence at a first end, a targeting payload sequence that hybridizes to a target sequence in a nucleic acid of interest, and a second orthogonal ligation adapter sequence at a second end, one or more signal oligonucleotides, each comprising in order a first orthogonal ligation adapter sequence at a first end, a signaling payload sequence that hybridizes to an imaging oligonucleotide, and a second orthogonal ligation adapter sequence at a second end, a first splint oligonucleotide with a first domain that hybridizes to the second orthogonal ligation adapter sequence of the targeting oligonucleotide and a second domain that hybridizes to the first orthogonal ligation adapter sequence of a first signal oligonucleotide: permitting the first splint oligonucleotide to hybridize to the second orthogonal ligation adapter sequence of the targeting oligonucleotide and the first orthogonal ligation adapter sequence of the first signal oligonucleotide, thereby bringing the second end of the targeting oligonucleotide and the first end of the first signal oligo into close proximity: ligating the second end of the targeting oligonucleotide to the first end of the first signal oligonucleotide to provide a ligated probe precursor.

Embodiment 38. The method of embodiment 37, further comprising attaching end adapters to each end of the ligated probe precursor, wherein each end adapter comprises a primer binding site.

Embodiment 39. The method of embodiment 37, wherein: the reaction mixture further comprises: a first terminal oligonucleotide, comprising a first orthogonal ligation adapter sequence at a first end and a primer payload sequence that comprises a first primer binding site, a second terminal oligonucleotide, comprising a first orthogonal ligation adapter sequence at a first end and a primer payload sequence that comprises a second primer binding site, wherein the first primer binding site and the second primer binding site are the same or different, a first end splint oligonucleotide with a first domain that hybridizes to the first orthogonal ligation adapter sequence of the first terminal oligonucleotide and a second domain that hybridizes to the first orthogonal ligation adapter sequence of the targeting oligonucleotide, and a second end splint oligonucleotide with a first domain that hybridizes to the first orthogonal ligation adapter sequence of the second terminal oligonucleotide and a second domain that hybridizes to the second orthogonal ligation adapter sequence of a signal oligonucleotide; and the hybridizing step further comprises: permitting the first end splint oligonucleotide to hybridize to the first orthogonal ligation adapter sequence of the first terminal oligonucleotide and the first orthogonal ligation adapter sequence of the targeting oligonucleotide, thereby bringing the first end of the first terminal oligonucleotide and the first end of the targeting oligonucleotide into close proximity; and permitting the second end splint oligonucleotide to hybridize to the first orthogonal ligation adapter sequence of the second terminal oligonucleotide and the second orthogonal ligation adapter sequence of a signal oligonucleotide, thereby bringing the first end of the second terminal oligo and the second end of the signal oligonucleotide into close proximity; and the ligating step further comprises: ligating the first end of the first terminal oligonucleotide to the first end of the targeting oligonucleotide; and ligating the first end of the second terminal oligonucleotide to the second end of the signal oligonucleotide.

Embodiment 40. The method as in any one of embodiments 37-39, further comprising amplifying the ligated probe precursor with primers that bind to the first primer biding site and the second primer binding site to provide an amplified probe precursor.

Embodiment 41. The method as in any one of embodiments 37-39, further comprising transcribing the amplified probe precursor to provide an RNA probe precursor.

Embodiment 42. The method of embodiment 41, further comprising reverse transcribing the RNA probe precursor to provide a single stranded DNA probe construct.

Embodiment 43. The method of embodiment 40, further comprising subjecting the amplified probe precursor to exonuclease digestion to provide a single stranded DNA probe construct.

Embodiment 44. The method of embodiment 40, further comprising subjecting the amplified probe precursor to DNA nicking and gel purification to provide a single stranded DNA probe construct.

Embodiment 45. The method as in any one of embodiments 37-39, further comprising performing circle-to-circle DNA amplification to provide a single stranded DNA probe construct.

Embodiment 46. The method as in any one of embodiments 37-45, wherein the method comprises incorporating a plurality of signal oligonucleotide into the ligated probe construct, wherein each additional signal oligonucleotide is incorporated by: providing at least one additional splint oligonucleotide with a first domain that hybridizes to the second orthogonal ligation adapter sequence of an adjacent oligonucleotide and a second domain that hybridizes to the first orthogonal ligation adapter sequence of the additional signal oligonucleotide, permitting the additional splint oligonucleotide to hybridize to the second orthogonal ligation adapter sequence of the adjacent oligonucleotide and the first orthogonal ligation adapter sequence of an additional signal oligonucleotide, thereby bringing the second end of the adjacent oligonucleotide and the first end of the additional oligonucleotide into close proximity; and ligating the second end of the adjacent oligonucleotide to the first end of the additional oligonucleotide to provide a ligated probe precursor.

Embodiment 46. The method of embodiment 45, comprising incorporating between 2 and 20 signal oligonucleotides.

Embodiment 47. The method of embodiment 46, wherein each of the plurality of signal oligonucleotides comprise a signaling payload sequence, wherein the signaling payload sequence hybridizes to a same or different imaging oligonucleotide, in any combination or order.

Embodiment 48. The method as in any one of embodiments 37-47, wherein the first orthogonal ligation adapter sequence of the targeting oligonucleotide and the one or more signal oligonucleotide are different.

Embodiment 49. The method as in any one of embodiments 37-48, wherein the second orthogonal ligation adapter sequences of the targeting oligonucleotide and the one or more signal oligonucleotides are different.

Embodiment 50. The method as in any one of embodiments 37-48, wherein the first orthogonal ligation adapter sequence and the second orthogonal ligation adapter sequence in any single domain are different.

Embodiment 51. A single stranded nucleic acid probe construct produced by the method recited in any one of embodiments 37-50.

Embodiment 52. A method of tagging a target nucleic acid of interest with a detectable signal, the method comprising: contacting the target nucleic acid of interest with a single stranded nucleic acid construct of any one of embodiments 1-17: contacting the single stranded nucleic acid construct with one or more imaging oligonucleotides; and providing conditions to allow the targeting payload sequence to hybridize to the target nucleic acid of interest and to allow the one or more imaging oligonucleotides to hybridize to one or more signaling payload sequences.

Embodiment 53. The method of embodiment 52, further comprising detecting signal produced by one or more imager oligos.

Embodiment 54. The method of embodiment 52, wherein the target nucleic acid of interest is a chromosomal DNA.

### EXAMPLES

The following examples are set forth so as to provide those of ordinary skill in the art with a complete disclosure and description of how to make and use the present invention and are not intended to limit the scope of what the inventors regard as their invention nor are they intended to represent that the experiments below are all or the only experiments performed.

### Example 1

The Example describes a new a multiplexed detection platform where amplification (1) produces pre-determined amplicons with a uniform and precisely known number of binding sites for fluorescent imager strands, (2) is fully programmable and modular, allowing the number of binding sites and resulting fluorescence intensity to be tuned precisely, (3) is free of sequence constraints, allowing for the introduction of arbitrary sequences of interest into product molecules, (4) is amenable to the latest multiplexing strategies (e.g. can yield chimeric products that facilitate combinatorial and/or ratiometric labeling), and (5) takes place in vitro as a highly scalable preparative step, to remove amplification-associated sources of variation from FISH-based methods. The disclosed approach can create designer molecular patterns in vitro, and further validate that the ssDNA molecules produced by the disclosed method lead to robust fluorescent signals when deployed in in situ hybridization reactions.

### Results

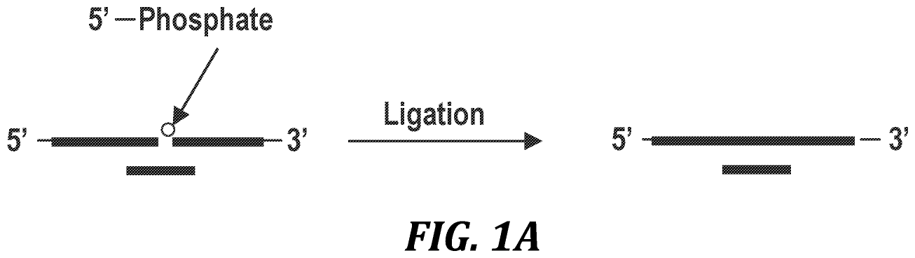

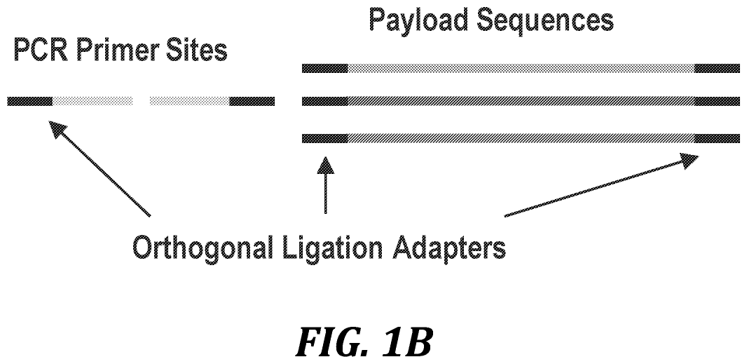

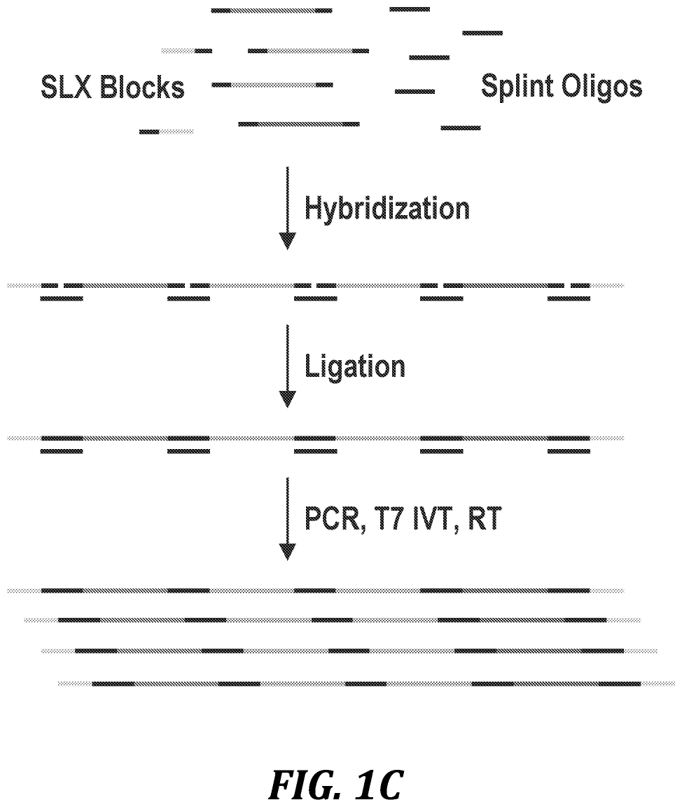

This Example describes the development of a new type of ssDNA probe reagent for use in FISH-based methods that is based on the power of splint ligation, wherein a ligation junction is created between two oligonucleotide termini through hybridization of a third complementary oligonucleotide and subsequently ligated (FIG. 1a). It was reasoned that an approach based on the multiplexed splint ligation of oligonucleotide ‘blocks’ containing payload sequences of interest flanked by orthogonal ligation adapter sequences (FIG. 1b) could produce arbitrary, modular ssDNA products from short oligonucleotides, and that the resulting ssDNA material could then be produced at scale using recent ssDNA amplification strategies. The resulting ssDNA products were termed splint ligation extension (SLX) probes.

To synthesize SLX probes, a multi-molecular ligation complex can be assembled from SLX building block oligonucleotides and corresponding splints by hybridization. The inclusion of particular splint sequences programs, which determine where ligation junctions will form, and therefore, determine the sequence of the resulting SLX probes. This complex is then ligated, producing the desired ssDNA product. To produce probes at scale, terminal PCR adapter sequences are added during ligation, and the full-length ligation product is selectively amplified using terminal PCR primers. A T7 promoter added during PCR renders the PCR product a valid template for T7 in vitro transcription (IVT), producing an RNA version of the SLX probes, which is reverse-transcribed to yield the final ssDNA SLX probes at scale (FIG. 1c). It will be appreciated that other methods can be used to amplify the ligation products and convert them back to ssDNA constructs and are similarly encompassed by this disclosure. For example, PCR can be followed by selective exonuclease digestion, PCR can be followed by DNA nicking and gel purification, and circle-to-circle DNA amplification can be performed.

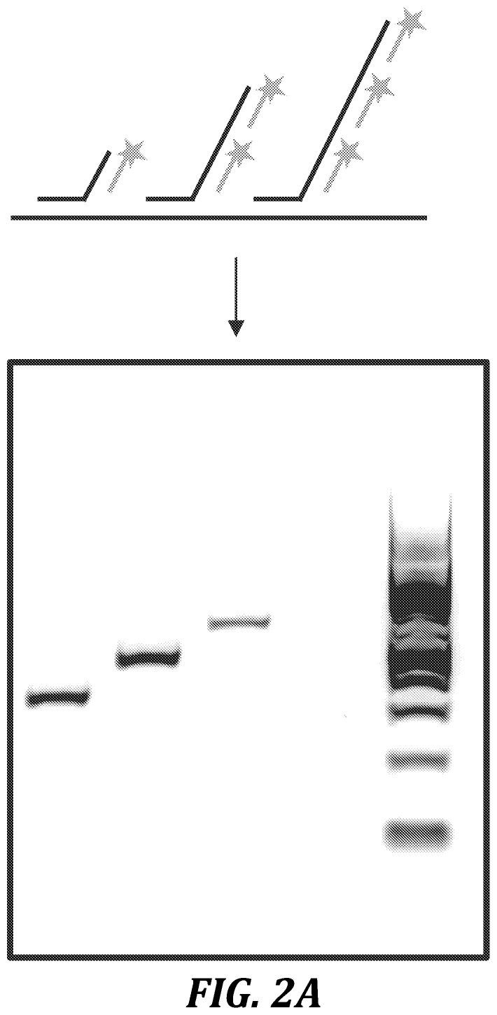

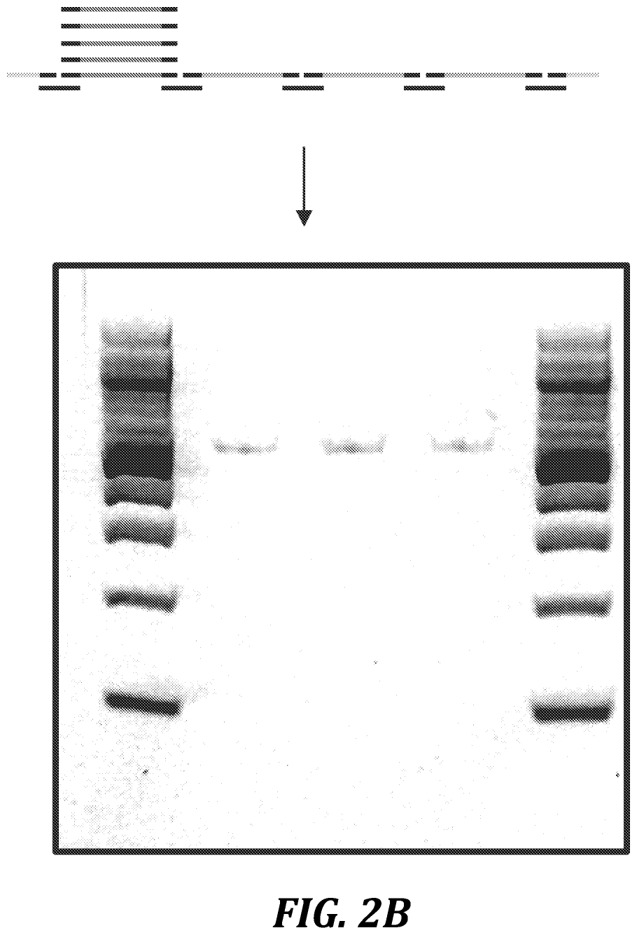

It was experimentally demonstrated that SLX probes can be synthesized with precise numbers of binding sites for fluorescent imager strands (FIG. 2a) and that multiple SLX probes can be synthesized in a one-pot reaction (FIG. 2b). Using Human metaphase spreads, DNA-FISH targeting the Human telomere repeat sequence using SLX probes and targeting specific regions on chromosome X with a pool of SLX probes in a multiplexed experiment was demonstrated. SLX probe synthesis is thus accessible and the probes have several desirable properties for in situ detection, making these reagents readily adoptable for quantitative FISH-based methods. For example, SLX-FISH constructs targeting the Telomere sequence, hg38, on Human metaphase spreads can be generated and SLX-FISH constructs targeting a primary probe library comprising 200-kb regions on Human chromosome X with 1000 primary probes per region can be generated. Probes can be synthesized by ligating several building block oligos: two terminal PCR primer adapter sites, a Human Telomere-targeting homology domain, and two blocks containing a readout sequence to be visualized using fluorescent secondary imager probes. DNA can be stained with DAPI. The SLX probes target the primary genomic probes and a bridging strategy is used to target the SLX probes to specific spots labeled by the primary library.

### Design of Orthogonal Ligation Adapter Sequences

In an effort to obtain desired ligation products while preventing unwanted side products resulting from cross-hybridization or non-specific ligation, a set of orthogonal adapter sequences were computationally designed with favorable thermodynamic properties for specific complex formation via hybridization and ligation at 25° C. Starting with all possible 10-mers (n=10⁶), sequences were excluded based on Tm, propensity to form secondary structure, homodimer Tm, 3′- and 5′-hairpin Tm, and self-complementarity. The remaining sequences were considered acceptable and from this set, a subset (n≈10³) was constructed with minimum Hamming distance constraints both overall and more stringent constraints near ligation junctions (adapter termini), with respect to other sequences in the set and also to their reverse complements and this set was used for all SLX probe synthesis experiments.

### SLX Probe Synthesis

The first step in synthesizing SLX probes is to form the multi-molecular ligation complex via hybridization. This complex was observed to form rapidly at room temperature in the hybridization buffer (2×SSC), with successful ligations performed as soon as 10 minutes after combining the 5′-phosphorylated SLX building block oligos and splints in a hybridization buffer. This complex is also stable and can be successfully ligated after storage at room temperature for at least several days. Similarly, it was found that enzymatic ligation with T4 DNA Ligase was rapid. Overnight ligations were also successful, and probe synthesis was successful independent of whether the ligation reaction was heat inactivated.

To amplify ligation products, two rounds of PCR were implemented. The first PCR is a low-cycle reaction which serves to double strand and enrich for full-length ligation products, as incomplete SLX probes will not bear both terminal primer sites. This reaction is diluted and used (unpurified) as the template for a second PCR, which serves to produce a sufficient quantity of DNA template for T7 in vitro transcription (IVT). The completion of the first PCR marks the end of the probe synthesis phase and the beginning of ssDNA production, as the PCR product is a renewable resource, and ligation will never need to be repeated for that particular SLX probe or probe pool. Production of ssDNA proceeded according to established ssDNA workflows (Murgha, Y. E., Rouillard, J.-M. & Gulari, E. Methods for the Preparation of Large Quantities of Complex Single-Stranded Oligonucleotide Libraries. PLoS One 9, e94752 (2014); Murgha, Y. et al. Combined in vitro transcription and reverse transcription to amplify and label complex synthetic oligonucleotide probe libraries. Biotechniques 58, 301-307 (2015)) namely the addition of a T7 promoter during the second PCR, then T7 IVT, followed by enzymatic DNA template degradation, and finally reverse transcription followed by enzymatic RNA template degradation yielding the final ssDNA probes. This ssDNA production workflow is arbitrarily scalable, making it convenient for scaling production up for large tissue experiments. For cell culture experiments, the standard workflow scale produces enough SLX probe reagents for many experiments.

The SLX probe synthesis and ssDNA production workflows produced the desired products and were free from undesired byproducts. As expected, uniform products are obtained rather than a population of products with different lengths, as are produced by many existing stochastic amplification strategies. This held true when ligation reactions of increased complexity were performed, whether to precisely modulate the number of imager binding sites (FIG. 2a), or to incorporate a pool of SLX block oligos at a single position instead of a single block sequence, which results in a pool of SLX probes (FIG. 2b). The SLX probes that are used in the multiplexed FISH demonstration were produced using such a pool of targeting block oligos as illustrated in a one-pot multiplexed ligation reaction (FIG. 2b).

DNA-FISH with SLX Probes

As a functional validation of synthesized SLX probes, DNA-FISH was performed against chromosomal targets on Human metaphase chromosome spreads. First, the telomere repeat sequence was targeted with a single SLX probe. To create the telomere SLX probe, a telomere-targeting block oligo was used in conjunction with several ‘payload’ blocks bearing repeats of a fluorescent imager strand binding sequence. The next experiment tested three SLX probe pools (one per fluorescent channel) each produced from one-pot multiplexed ligation of several targeting blocks attached to common payload blocks. A synthetic library of primary genome-binding probes targets 200-Mb regions (“spots”) spanning Human chromosome X with 1000 probes per spot, and each spot bearing a unique bridge sequence. The synthesized SLX probe pools target the bridge sequences on the primary probes, so each spot is addressable in any color, and color patterns are programmed by including or excluding particular targeting and payload block oligos and splints during SLX probe synthesis.

### DISCUSSION

Leveraging a multiplexed splint ligation approach with ssDNA amplifications strategies, the method referred to herein as ‘splint ligation extension’ (SLX) enables the modular synthesis of programmable ssDNA probes at scale. The SLX probe synthesis workflow was demonstrated and applied SLX probes to DNA-FISH experiments in situ. The unique properties of SLX probes have broad utility for FISH-based technologies. For instance, the ability to modulate intensity by precisely controlling the number of binding sites for fluorescent imager strands allows intensity to be increased as needed for imaging deep within tissues, or low abundance targets, or for shorter imaging exposure times to increase imaging throughput. Because SLX block oligo payload sequences are not directly involved in probe synthesis, they are relatively unconstrained, allowing arbitrary sequences to be introduced into ssDNA probes. Additionally, the deterministic nature of SLX probe synthesis removes the variation introduced by performing amplification within each biological sample.

Given the modular nature of SLX probes, it is anticipated they will naturally facilitate intensity barcoding, ratiometric labeling, and combinatorial labeling strategies for increasing multiplexity, as well as combinations of these approaches. It is expected that the tunable number of binding sites and the fact that a programmed number of sites is obtained precisely, rather than a polydisperse blend of product lengths, reagents can be generated to give signals that are easily resolvable into discrete levels of signal intensity, which enables the use of signal intensity as an additional channel to transmit information. This would increase the information content of each image, leading to higher throughput and less imaging data which is slow and costly to acquire, store, and analyze.

The SLX probe synthesis workflow is versatile and can be adapted to many in situ technologies with only slight modifications. For instance, by performing the last step (reverse transcription) with a fluorescently-labeled reverse transcription primer, fluorescent SLX probes are obtained, obviating the need for secondary imager probes. On the other hand, if an Acrydite-modified reverse transcription primer is used, then the resulting SLX probes are functionalized with tethering moieties rendering them compatible with technologies related to Expansion Microscopy (Chen, F., Tillberg, P. W. & Boyden, E. S. Expansion microscopy. Science (80). 347, 543-548 (2015)) and ExFISH (Chen, F. et al. Nanoscale imaging of RNA with expansion microscopy. Nat. Methods 13, 679-684 (2016)). Countless other labels, chemical modifications, and chemistries could be introduced in this way. The disclosed SLX probes can be broadly applied in the context of existing and novel in situ detection technologies in research and clinical diagnostics.

### Methods

### SLX Probe Synthesis

All SLX building block oligonucleotides and splints were ordered from Integrated DNA Technologies. Block oligos to be ligated were ordered with 5′-phosphate modification, with the exception of the left-most block which always adds the 5′-terminal PCR primer adapter. To form the multi-molecular ligation complex, selected block and splint oligos were combined in an equimolar ratio at 1 μM final each in 2×SSC as a hybridization buffer. Complexes were incubated at room temperature from 10 minutes to overnight. One microliter of the 1 μM complex was ligated in a standard 20 μL T4 DNA Ligase (NEB) reaction for 10 minutes to overnight. After ligation (and optionally, heat inactivation), a 1:10 dilution of the ligation is made with dH2O, and one microliter of this dilution is the input to the first PCR. The first PCR mix contained 34 μl dH2O, 10 μl 5× Phusion HF Buffer, 1.5 μl 10 mM dNTP Mix, 1.5 μl 10 μM F primer, 1.5 μl 10 μM R primer, 1.0 μl diluted ligation reaction, 0.5 μl Phusion DNA Polymerase (2 U/μl) for a total volume of 50 μl. The thermal cycler program comprised an initial denaturation at 95° C. for 3 minutes, followed by 12 cycles of 98° C. for 20 seconds, 60° C. for 15 seconds, 72° C. for 15 seconds, and a final extension at 72° C. for 1 minute followed by a 4° C. hold. The first PCR is diluted 1:1000 with dH2O and that material is stored at −20 C indefinitely and used for ssDNA production as needed.

ssDNA Production

The second PCR mix contained 27 μl dH2O, 10 μl 5× Phusion HF Buffer, 1.5 μl mM dNTP Mix, 5.0 μl 10 μM F Primer, 5.0 μl 10 μM R Primer, 1.0 μl diluted DNA template, 0.5 μl Phusion DNA Polymerase (2 U/μl) for a total volume of 50 μl. The thermal cycler program was the same as before but with 18 cycles instead of 12. To purify the second PCR product, 0.1 volumes of 5 M Ammonium acetate, 0.02 volumes of 2% (wt/vol) Glycogen, and 3.0 volumes of 100% (vol/vol) Ethanol were added to the reaction mixture. The resulting mixture was incubated for 15 minutes at −20° C., followed by 10 minutes of centrifugation at 10,000×g at 4° C. The pellet was washed using 750 ul of 70% (vol/vol) and centrifuged again as before. The pellet was dried for 3 minutes at room temperature and resuspended using 250 μl nuclease free water. The concentration was measured using a NanoDrop spectrophotometer. RNA was synthesized using the NEB HiScribe T7 Quick High Yield RNA Synthesis Kit with a modified reaction mix containing 8 μl dH2O, 2.5 μl diluted DNA template, 15.0 μl NTP Buffer Mix, 3 μl T7 RNA Polymerase Mix, 1.5 μl RNaseOUT. The reaction was incubated at 37° C. overnight. Enzymatic digestion of the DNA template and precipitation of the RNA using the included Lithium chloride solution were both carried out according to the manufacturer's standard protocol. The reverse transcription reaction contained 55 μl synthesized RNA, 30 μl 5× RT Buffer, 48 μl 10 mM dNTP Mix, 10 μl 100 μM RT Primer, 3 μl RNaseOUT, and 4 μl Maxima H Minus Reverse Transcriptase (200 U/μl) for a total volume of 150 μl, which was split into four 37.5 μl reactions. The reactions were incubated at 50° C. for 2 hours and then at 80° C. for 5 minutes. RNA templates were degraded enzymatically by adding 1 μl RNase to each reaction and incubating at 37° C. for 1 hour. To precipitate the final ssDNA probes, 0.1 volumes of 5 M Ammonium acetate, 0.02 volumes of 2% (wt/vol) Glycogen, and 3.0 volumes of 100% (vol/vol) Ethanol were added to the reverse transcription reaction mixture. The resulting mixture was incubated for 15 minutes at −20° C., followed by 10 minutes of centrifugation at 10,000×g at 4° C. The pellet was washed using 750 ul of 70% (vol/vol) and centrifuged again as before. The pellet was dried for 3 minutes at room temperature and resuspended using 250 μl nuclease free water. The concentration was measured using a NanoDrop spectrophotometer and a 10 μM probe stock solution was prepared and stored at −20° C.

DNA-FISH with SLX Probes

DNA-FISH on Human metaphase chromosome spreads was carried out as previously described (Kishi, J. Y. et al. SABER amplifies FISH: enhanced multiplexed imaging of RNA and DNA in cells and tissues. Nat. Methods 16, 533-544 (2019)). Briefly, 60 ρmol total of SLX primary probe targeting human alpha satellite (pan alpha experiment) and 60 ρmol of SLX secondary “bridge” probe mix+60 ρmol amplified ssDNA primary probes from an existing 30-target chrX library (Hershberg, E. A., et al. PaintSHOP enables the interactive design of transcriptome- and genome-scale oligonucleotide FISH experiments. Nat Methods (2021)) were dried using a SpeedVac concentrator. The dried oligos were resuspended using 25 μl of an ISH solution containing 12.5 μl Formamide, 5.0 μl 50% Dextran sulfate, 4.0 μl dH2O, 2.5 μl 20×SSC, 1.0 μl RNase A (10 mg/ml). Human metaphase chromosome spreads (XX 46N, Applied Genetics Laboratories) were denatured in 70% (vol/vol) Formamide in 2×SSCT (2×SSC with 1% (vol/vol) Tween-20) at 70° C. (90 seconds) and then transferred to ice-cold 70% (vol/vol) ethanol (5 minutes), to 90% (vol/vol) ethanol (5 minutes), and to 100% ethanol (5 minutes). Slides were air dried and the primary hybridization mix was added and sealed underneath a coverslip with rubber cement. Slides were placed in a humidified chamber and incubated in an oven at 37° C. overnight. After hybridization, coverslips were removed and slides were washed in 2×SSCT at 60° C. (15 minutes) and in 2×SSCT at room temperature (2× 5 minutes). A 25 μl secondary hybridization solution comprising 11.0 μl dH2O, 5.0 μl 5×PBS-T (5×PBS with 0.5% (vol/vol) Tween-20), 5.0 μl 50% Dextran sulfate, and 4.0 μl 10 μM total fluorescently labeled secondary oligos. The secondary hybridization mix was added and sealed underneath a coverslip with rubber cement. Slides were placed in a humidified chamber and incubated in an oven at 37° C. (1 hour). After hybridization, coverslips were removed and slides were washed in 1×PBS-T (1×PBS with 0.1% (vol/vol) Tween-20) at 37° C. (3×15 minutes). Slides were imaged on a custom microscopy system consisting of a Nikon Eclipse Ti2 body and an attached Yokogawa CSU-WI SoRa spinning disc confocal unit. 405 nm, 488 nm, 561 nm, or 640 nm laser excitation was emitted at 30-40% of maximal intensity inside of a Nikon LUNF 405/488/561/640NM IF commercial launch and coupled into a single-mode optical fiber, which delivered the excitation light into the CSU-WI SoRa unit. Excitation light was then directed through a micro-lens array disc and a ‘SoRa’ disc containing 50 μm pinholes and directed to the rear aperture of a 100× N.A. 1.49 Apo TIRF oil immersion objective lens by a prism in the base of the Ti2. Emission light was collected by the same objective and passed via a prism in the base of the Ti2 back into the SoRa unit, where it was relayed by a 1× lens (fields of view) or 2.8× lens (spreads) through the pinhole disc and then directed into the emission path by a quad-band dichroic mirror (Semrock Di01-T405/488/568/647-13×15×0.5). Emission light was then spectrally filtered by one of four single-bandpass filters (DAPI: Chroma ET455/50M; ATTO 488: Chroma ET525/36M; ATTO 565: Chroma ET605/50M; Alexa Fluor 647: Chroma ET705/72M) and focused by a 1× relay lens onto an Andor Sona 4.2B-11 camera with a physical pixel size of 11 μm, resulting in an effective pixel size of 110 nm (fields of view) or 39.3 nm (spreads). The Sona was operated in 16-bit mode with rolling shutter readout and a 300 ms exposure time. Acquisition was controlled by Nikon Elements software. Images were processed in ImageJ and Adobe Photoshop.

### Example 2

This Example provides additional discussion regarding development and use of the SLX probes.

### Background

Fluorescence in situ hybridization (FISH) and immunofluorescence (IF) enable the interrogation of the location and abundance of target nucleic acid sequences and proteins, respectively, in fixed cells and tissues and have enabled the development of many advanced biomolecular profiling technologies for the multiplexed in situ localization of RNAs (‘spatial transcriptomics’), DNA loci (‘spatial genomics’), and proteins (‘spatial proteomics’). These methods leverage the specificity of nucleic acid base pairing and antibody-antigen interactions to anchor fluorescent molecules at the sites of their molecular targets. Such techniques have broad applications in basic science research, translational research, clinical trials, and medical diagnostics.

Existing technologies employ a diverse set of strategies to create an amplified signal that can be detected robustly at the site of the target molecule. For FISH-based technologies, signal amplification usually involves introducing additional ssDNA binding sites where the fluorescent probe can hybridize. There are enzymatic, hybridization-based, and chemical strategies for amplification of the target ssDNA, including Rolling Circle Amplification (RCA), Primer Exchange Reaction (PER), and Hybridization Chain Reaction (HCR), among others. A common attribute of these reactions is that due to stochastic reaction dynamics, a polydisperse blend of different-length products is generated. While this accomplishes the goal of making fluorescent puncta bright enough to be detected, it also introduces non-uniformity into the signal intensities of fluorescent puncta that is unrelated to target abundance.

This disclosure provides Splint Ligation Extension (SLX) probes, which are ssDNA probe reagents with a known length and sequence, synthesized via splint ligation of modular “building block” oligos, and produced at scale using recent ssDNA amplification techniques (Minev, D. Nucleic Acids Res. 47, 11956-11962 (2019); Murgha, Y. Biotechniques 58, 301-307 (2015), each of which is incorporated herein by reference in its entirety). SLX probe reagents can be used in RNA FISH, DNA FISH, and IF experiments and have desirable properties over existing FISH-based probe technologies and can be used to augment these methods. In contrast to existing technologies, the number of binding sites for secondary fluorescent oligo probes is known as it is programmed during synthesis and results in uniform probe molecules. This affords SLX-FISH technologies with absolute quantification capability with respect to target molecule abundance. The precise tuning of signal intensity by modulating binding site abundance also enables the use of signal intensity as an additional channel to transmit information, which increases the information content of each image, leading to higher throughput and less imaging data which is slow and costly to acquire, store, and analyze.

### Description of the SLX Platform

SLX probes are oligonucleotide reagents synthesized through splint ligation and subsequent ssDNA amplification. Desired probe constructs are obtained by mixing “building block” oligos with complementary splint oligos that hybridize to building blocks, bringing them in proximity and forming ligation junctions. Building block oligos contain “payload” sequences of interest flanked by orthogonal ligation adapter sequences. Payload sequences are arbitrary functional sequence domains that afford SLX probes with desired functionality (e.g., a genome-targeting homology domain, a binding site for a fluorescent imager strand, a PCR priming site, etc.). Ligation adapters are computationally designed to prevent non-specific ligation products. Because adding a splint oligo to the mix programs a particular ligation junction which will link two building blocks, the synthesis of arbitrary complex and feature-rich SLX probes is programmed through the inclusion of particular splint oligos.

Once the multi-molecular ligation complex is assembled and ligated, SLX probes can be amplified and produced at scale. PCR is used to selectively amplify complete ligation products as only molecules containing both terminal PCR priming sites will amplify. A T7 promoter is introduced on the reverse PCR primer, which creates a suitable template for RNA synthesis via T7 in vitro transcription. Finally, this RNA is reverse-transcribed and the RNA template is chemically or enzymatically degraded, yielding the finished SLX probe reagents, which are easily purified via Ethanol precipitation. This workflow is arbitrarily scalable, making it convenient to produce SLX probe reagents for use in large-scale tissue experiments.

For example, modular SLX building block oligos and complementary splints can hybridize to form a multi-molecular SLX ligation complex. Additionally, a complex can be formed when two oligos hybridize adjacently to a complementary “splint” oligo that has homology to both oligos. T4 DNA Ligase then ligates the adjacent oligos, forming a new longer oligo product. First, a complex can be formed via hybridization of “building block” oligos and complementary splints whose sequences determine which ligation junctions will form. This complex is ligated forming a new long product which is amplified by PCR, followed by in vitro transcription and then reverse transcription, yielding the final SLX probe material.

In contrast to existing in situ omics technologies, which rely on stochastic amplification steps that produce non-uniform distributions of available binding site abundance for secondary fluorescent imager strands, SLX probe reagents contain a known, uniform number of binding sites, and this number is precisely tunable. For example, the signal intensity that a particular probe will produce can be precisely controlled by tuning the number of binding sites available for a fluorescent secondary oligo probe to bind during imaging. This enables the usage of signal intensity as an additional channel for encoding information, which increases throughput. With calibration, absolute quantification of target abundance can be performed using SLX probe reagents. This property can be leveraged to transform one of the many qualitative detection schemes into quantitative detection technologies, increasing their power.

Quantification of target abundance as measured through signal intensity can be achieved, e.g., using a one-color and multiple-color modality, or multiple channels/modalities. One color is sufficient for quantitative measurements as there is no variance introduced by polydispersity of probe lengths, so fluorescence intensity is expected to be proportional to target abundance, as there is a deterministic number of available binding sites for fluorescent imager probe oligos. The use of two colors enables the use of intensity “bins” (distinct levels of intensity) as a new channel in which information can be encoded, which is currently not utilized by the existing methods. For instance, instead of encoding two targets using “red” and “green” fluorescent channels, the targets could be labeled with two different intensity levels within the “red” channel, increasing the number of targets that can be simultaneously detected in one imaging cycle, which increases multiplexity and lowers reagent and data storage costs, as well as imaging and analytical compute times, providing superior detection capability per unit cost. This property of SLX probe reagents can be leveraged to increase throughput of existing in situ omics technologies.

SLX probe reagents are modular and flexible: several variants of the synthesis workflow introduce diverse functionalities into SLX reagents. For instance, by performing the last step (reverse transcription) with a fluorescently-labeled reverse transcription primer, fluorescent SLX probes are obtained, obviating the need for secondary imager probes. On the other hand, if an Acrydite-modified reverse transcription primer is used, then the resulting SLX probes are functionalized with tethering moieties rendering them compatible with technologies related to Expansion Microscopy (Chen, F. Science (80) 347, 543-548 (2015)) and ExFISH (Chen, F. Nat. Methods 13, 679-684 (2016)). Other embodiments involve performing chemical ligations in lieu of enzymatic ligations, using PCR-based amplification workflows in the place of in vitro transcription, or performing ligations and subsequent purifications at scale without downstream amplification, or diffusing small oligos into tissues and performing ligation in situ, among others. The workflow, like the reagents themselves, are highly modular.

### Discussion: Advantages and Applications

SLX probe reagents contain precisely programmed numbers of binding sites for secondary fluorescent imager probes. This allows the signal intensity to be tuned per target; for example, targets for which only a few homology probe sequences are available may benefit from longer SLX probes, increasing signal intensity, whereas very abundant targets or targets with many more available probes may use shorter SLX probes to obtain relatively balanced signal intensities across a wide range of targets with diverging abundance.

SLX probes can also be readily produced at scales of 20-50× greater than the field standard ‘SABER’ method, making it uniquely able to produce a known signal amplification substrate in vitro at scale than can be QC′d and then added to the in situ experiment.

Quantitative labeling can be achieved with SLX probe reagents either through calibration to standards or internal normalization using a ratio of signal intensities in two channels. The tunable number of binding sites and the fact that a programmed number of sites is obtained precisely, rather than a polydisperse blend of product lengths, reagents can be generated to give relatively discrete levels of signal intensity, which allows the use of intensity as an additional channel for encoding information. Conceptually analogous improvements have been made to Illumina's next-generation sequencing to accomplish four-base discrimination using only two fluorescent channels. Adding this additional information channel increases the number of targets that can be imaged simultaneously, and/or reduces the number of channels/imaging cycles needed to capture a given number of targets. This increases throughput, and decreases the cost of reagents, imaging time, and imaging file size footprint for a given experiment, simply by substituting SLX probe reagents for the reagents currently utilized by existing methods.

### Example 3

Use of exonucleases to remove precursors and incomplete ligation products after the ligation step.

The goal is to select for complete ligation products (i.e., containing 100% of intended SLX ligation block oligos, having been ligated at every ligation junction within the ligation complex). Amplification via PCR is already selective for complete ligation products by means of requiring both of the termin al PCR primer binding sites added during ligation to achieve exponential amplification, and incomplete ligation products necessarily lack at least one terminal PCR primer binding site. Additional purification steps may be desirable to enable ligation at scale (i.e., without downstream PCR amplification) and/or to produce cleaner PCR templates prior to amplification, etc.

Two independent, directional strategies (i.e., employing both 5′ to 3′ and 3′ to 5′ exonuclease activities to digest precursor molecules and incomplete ligation products while complete ligation products are protected) are used in conjunction in this example; in other embodiments, either of the strategies is used alone. In the 3′ to 5′ strategy, the 3′-most block oligo is protected from 3′ exonuclease activity. In the 5′ strategy, the 5′-most block is protected from 5′ exonuclease activity. In both strategies, oligos ligated to the protect oligo are afforded the same protection: when the strategies are combined, only complete ligation products are protected from digestion.

### Implementation

Add a 5′-phosphorothioate (PS) modification to all splint oligos, so that Lambda Exonuclease degrades them. Every ligation block except for the 5′ most block already requires 5′-phosphate modification for ligation. Following this, the result is that the 5′-most block oligo is protected from lambda exonuclease digestion, while all other oligos would be degraded. When used in conjunction with a protected 3′ block and Exonuclease i and Exonuclease iii, only a complete ssDNA ligation product is protected from all exonucleases (e.g., Lambda Exonuclease, Exonuclease I, and Exonuclease III), while partial ligation products, partial synthesis products, and phosphorylated splints will be degraded.

### Protective Modification

Protection from 3′ exonuclease activity is afforded to the 3′-most ligation block oligo through the addition of chemical modification during oligo synthesis. The phosphorothioate (PS) bond substitutes a sulfur atom for a non-bridging oxygen in the phosphate backbone of an oligo. This modification renders the internucleotide linkage resistant to nuclease degradation. Phosphorothioate bonds can be introduced between the last 3-5 nucleotides at the 5′- or 3′-end of the oligo to inhibit exonuclease degradation. Including phosphorothioate bonds throughout the entire oligo will help reduce attack by endonucleases as well. See e.g., Integrated DNA Technologies website.

### Workflow

To form SLX ligation complexes, all oligos (5′-phosphorylated ligation “block” oligos and splint oligos) are duplexed at a final concentration of 1 μM in 2×SSC buffer. The 3′-most “block” oligo—whose 3′ end becomes the 3′ end of the ligation product—is protected from exonuclease digestion using phosphorothiate linkages. See e.g., Integrated DNA Technologies website.

To ligate complexes, 1 μL of 1 μM ligation complex was added to a standard 20 μL T4 DNA Ligase reaction. For example:
16 μL dH2O;
2 μL 10× T4 DNA Ligase reaction buffer;
1 μL 1 μM ligation complex; and
1 μL T4 DNA Ligase;
20 μL total volume.

The ligation reaction was incubated for 10 minutes at room temperature, and 1 L of ligation reaction was put into an exonuclease digestion reaction:
14 μL dH2O Nuclease-free H2O;
2 μL 10×NEB CutSmart buffer or similar;
1 μL Ligation reaction;
1 μL Lambda Exonuclease;
1 μL Exonuclease I; and
1 μL Exonuclease III;
20 μL total volume.

The exonuclease digestion reaction was incubated at 37° C. for 30 minutes followed by heat inactivation at 80° C. for 20 minutes. Following this, 1 μL of the exonuclease reaction is then used in PCR.

Alternative Strategy for Amplifying SLX Probes after Ligation

Another approach to protecting ligation precursors and incomplete ligation products involves ligating a circular (i.e., as opposed to linear) ligation complex producing a closed ssDNA circle lacking 3′ and 5′ termini, thereby protecting the complete ligation product from exonuclease activity. The circular ligation product can then be cleaved by restriction enzymes or cis- or trans-acting DNAzymes, etc., producing a linear ssDNA product for downstream PCR amplification. Alternatively, in an approach that bypasses PCR, the circular ssDNA is amplified by Rolling Circle Amplification (RCA) and RCA products are cleaved (i.e., between concatemeric repeats) yielding the desired ssDNA SLX probe at scale.

While illustrative embodiments have been illustrated and described, it will be appreciated that various changes can be made therein without departing from the spirit and scope of the invention.

1. A single stranded nucleic acid construct with a predetermined number of binding sites, the construct comprising:
a targeting domain, comprising at least one programmable unit with each unit comprising from a 5′ end to a 3′ end, a first orthogonal ligation adapter sequence, a target payload sequence comprising a predetermined number of binding sites, and a second orthogonal ligation adapter sequence;
a signaling domain, comprising at least two programmable units with each unit comprising from a 5′ end to a 3′ end, a first orthogonal ligation adapter sequence, a signaling payload sequence comprising a predetermined number of binding sites, and a second orthogonal ligation adapter sequence; and
at least one splint oligonucleotide that hybridizes to a complementary orthogonal ligation adapter sequence on a first unit of a targeting domain and hybridizes to a complementary orthogonal ligation adapter sequence on a second unit of a signaling domain to form a ligation junction to link the first programmable unit with the second programmable unit to form a functional unit,
wherein the construct can have two or more functional units comprising the targeting domain of interest and at least one signaling domain of interest, and wherein the construct is amplified to produce the final construct product.

2. The construct of claim 1, wherein the targeting payload sequence hybridizes to a target sequence in a nucleic acid of interest, and wherein the signaling payload sequence hybridizes to an imaging oligonucleotide of interest.

3. (canceled)

4. The construct of claim 1, wherein the targeting domain comprises one or more functional sequences encoding one or more functional linkers, adapters, barcode tags, or other functional domains, in any order or combination, wherein the one or more sequences are disposed in a location between the first orthogonal ligation adapter sequence and the second orthogonal ligation adapter sequence.

5. The construct of claim 1, further comprising:
a terminal domain, comprising a first orthogonal ligation adapter sequence and a primer payload sequence that comprises a primer binding site, and
wherein a first terminal domain is covalently joined to the orthogonal ligation adapter sequence at the 5′ end of the construct and a second terminal domain is covalently joined to the orthogonal ligation adapter sequence at the 3′ end of the construct, wherein the terminal domain allows for amplification of the construct.

6. The construct of claim 1, comprising a plurality of signal domains, wherein a second and optionally a third, fourth, or fifth, signal domain comprises the signaling payload sequence, wherein the respective signaling payload sequence hybridizes to a same or different imaging oligonucleotide of interest, in any combination or order.

7. The construct of claim 1, wherein;
(i) the first orthogonal ligation adapter sequences of the targeting domain and the one or more signal domains are different;
(ii) the second orthogonal ligation adapter sequences of the targeting domain and the one or more signal domains are different; or
(iii) the first orthogonal ligation adapter sequence and the second orthogonal ligation adapter sequence in any single domain are different.

8-9. (canceled)

10. The construct of claim 1, wherein the signaling payload sequence comprises two or more binding sites, and wherein; (i) the binding sites are identical; or (ii) each binding site is different.

11-14. (canceled)

15. The construct of claim 1, wherein the construct further comprises a modified 5′ end and a modified 3′ end, wherein the modified 5′ end and the modified 3′ end protect the construct from exonuclease digestion.

16. The construct of claim 1, wherein the nucleic acid of interest comprises DNA and/or RNA.

17. The construct of claim 1, wherein the imaging oligonucleotide of interest is a fluorophore.

18. A method of quantifying a target nucleic acid, the method comprising:
(a) generating a first nucleic acid construct as recited in claim 1 for targeting a nucleic acid of interest, wherein the construct comprises a predetermined number of binding sites for an imaging oligonucleotide;
(b) generating a second nucleic acid construct for targeting a reference nucleic acid population, wherein the second construct comprises the same number of binding sites for the imaging oligonucleotide as the first construct generated in step (a);
(c) contacting the target nucleic acid of interest with the first construct, wherein the first construct hybridizes to the nucleic acid of interest;
(d) contacting the reference nucleic acid population with the second construct, wherein the second construct hybridizes to the reference nucleic acid population;
(e) adding the imaging oligonucleotide to saturate the binding sites on the first construct and the second construct;
(f) measuring the signal intensity from the imaging oligonucleotide bound to the first construct and the second construct; and
(g) comparing the signal intensity from the imaging oligonucleotide bound to the first construct to the signal intensity from the imaging oligonucleotide bound to the second construct, wherein a lower signal intensity indicates the target nucleic acid is present at a lower level compared to the reference nucleic acid population, and wherein a higher signal intensity indicates the target nucleic acid is present at a higher level compared to the reference nucleic acid population.

19. The method of claim 18, wherein the second nucleic acid construct comprises:
a targeting domain, comprising at least one programmable unit with each unit comprising from a 5′ end to a 3′ end, a first orthogonal ligation adapter sequence, a target reference nucleic acid payload sequence comprising a predetermined number of binding sites, and a second orthogonal ligation adapter sequence;
a signaling domain, comprising at least two programmable units with each unit comprising from a 5′ end to a 3′ end, a first orthogonal ligation adapter sequence, a signaling payload sequence comprising a predetermined number of binding sites that match the first construct, and a second orthogonal ligation adapter sequence; and
at least one splint oligonucleotide that hybridizes to a complementary orthogonal ligation adapter sequence on a first unit and hybridizes to a complementary orthogonal ligation adapter sequence on a second unit to form a ligation junction to link the first programmable unit with the second programmable unit to form a functional unit,
wherein the construct can have two or more functional units comprising the targeting domain of interest and at least one signaling domain of interest, and wherein the construct is amplified to produce the final construct product.

20. A multiplexed target detection method, the method comprising,
(a) combining a plurality of single stranded nucleic acid constructs each with a predetermined number of binding sites for imaging oligonucleotides with a plurality of splint oligonucleotides, wherein the constructs each comprise:
(i) a targeting domain, comprising at least one programmable unit with each unit comprising from a 5′ end to a 3′ end, a first orthogonal ligation adapter sequence, a target payload sequence comprising a predetermined number of binding sites, and a second orthogonal ligation adapter sequence; and
(ii) a signaling domain, comprising at least two programmable units with each unit comprising from a 5′ end to a 3′ end, a first orthogonal ligation adapter sequence, a signaling payload sequence comprising a predetermined number of binding sites, and a second orthogonal ligation adapter sequence;
and wherein the at least one splint oligonucleotide hybridizes to a complementary orthogonal ligation adapter sequence on a first unit and hybridizes to a complementary orthogonal ligation adapter sequence on a second unit to form a ligation junction to link the first programmable unit with the second programmable unit to form a first reaction mixture comprising a plurality of functional units;
(b) combining the first reaction mixture produced in step (a) with a sample containing a plurality of nucleic acid targets, wherein the functional units in the first reaction mixture produced in step (a) each hybridize with a nucleic acid target through the targeting domain producing a second reaction mixture;
(c) combining the second reaction mixture produced in step (b) with a plurality of imaging oligonucleotides, wherein the functional units in the second reaction mixture produced in step (b) each hybridize with an imaging oligonucleotide through the signaling domain; and
(d) imaging the labeled functional units.

21. The method of claim 20, wherein the targeting payload sequence of the functional unit hybridizes to a sequence in the nucleic acid target, and wherein the signaling payload sequence hybridizes to the imaging oligonucleotide.

22. (canceled)

23. The method of claim 20, wherein the targeting domain further comprises one or more functional sequences encoding one or more functional linkers, adapters, barcode tags, or other functional domains, in any order or combination, wherein the one or more sequences are disposed in a location between the first orthogonal ligation adapter sequence and the second orthogonal ligation adapter sequence.

24. The method of claim 20, further comprising:
a terminal domain, comprising a first orthogonal ligation adapter sequence and a primer payload sequence that comprises a primer binding site, and
wherein a first terminal domain is covalently joined to the orthogonal ligation adapter sequence at the 5′ end of the construct and a second terminal domain is covalently joined to the orthogonal ligation adapter sequence at the 3′ end of the construct, wherein the terminal domain allows for amplification of the construct.

25. The method of claim 20, comprising a plurality of signal domains, wherein a second and optionally third, fourth, or fifth, signal domain comprises the signaling payload sequence, wherein the signaling payload sequence hybridizes to a same or different imaging oligonucleotide, in any combination or order.

26. The method of claim 20, wherein;
(i) the first orthogonal ligation adapter sequences of the targeting domain and the one or more signal domains are different;
(ii) the second orthogonal ligation adapter sequences of the targeting domain and the one or more signal domains are different; or
(iii) the first orthogonal ligation adapter sequence and the second orthogonal ligation adapter sequence in any single domain are different.

27-28. (canceled)

29. The method of claim 20, wherein the signaling payload sequence comprises two or more binding sites, and wherein; (i) the binding sites are identical; or (ii) each binding site is different.

30-33. (canceled)

34. The method of claim 20, wherein the first construct and/or the second construct further comprise a modified 5′ end and a modified 3′ end, wherein the modified 5′ end and the modified 3′ end protect the first construct and/or the second construct from exonuclease digestion.

35-36. (canceled)

37. A method of assembling a single stranded nucleic acid probe construct, comprising:
providing reaction mixture comprising:
a targeting oligonucleotide, comprising in order a first orthogonal ligation adapter sequence at a first end, a targeting payload sequence that hybridizes to a target sequence in a nucleic acid of interest, and a second orthogonal ligation adapter sequence at a second end,
one or more signal oligonucleotides, each comprising in order a first orthogonal ligation adapter sequence at a first end, a signaling payload sequence that hybridizes to an imaging oligonucleotide, and a second orthogonal ligation adapter sequence at a second end,
a first splint oligonucleotide with a first domain that hybridizes to the second orthogonal ligation adapter sequence of the targeting oligonucleotide and a second domain that hybridizes to the first orthogonal ligation adapter sequence of a first signal oligonucleotide;
permitting the first splint oligonucleotide to hybridize to the second orthogonal ligation adapter sequence of the targeting oligonucleotide and the first orthogonal ligation adapter sequence of the first signal oligonucleotide, thereby bringing the second end of the targeting oligonucleotide and the first end of the first signal oligo into close proximity;
ligating the second end of the targeting oligonucleotide to the first end of the first signal oligonucleotide to provide a ligated probe precursor.

38. The method of claim 37, further comprising attaching end adapters to each end of the ligated probe precursor, wherein each end adapter comprises a primer binding site.

39. The method of claim 37, wherein:
the reaction mixture further comprises:
a first terminal oligonucleotide, comprising a first orthogonal ligation adapter sequence at a first end and a primer payload sequence that comprises a first primer binding site,
a second terminal oligonucleotide, comprising a first orthogonal ligation adapter sequence at a first end and a primer payload sequence that comprises a second primer binding site, wherein the first primer binding site and the second primer binding site are the same or different,
a first end splint oligonucleotide with a first domain that hybridizes to the first orthogonal ligation adapter sequence of the first terminal oligonucleotide and a second domain that hybridizes to the first orthogonal ligation adapter sequence of the targeting oligonucleotide, and
a second end splint oligonucleotide with a first domain that hybridizes to the first orthogonal ligation adapter sequence of the second terminal oligonucleotide and a second domain that hybridizes to the second orthogonal ligation adapter sequence of a signal oligonucleotide; and
the hybridizing step further comprises:
permitting the first end splint oligonucleotide to hybridize to the first orthogonal ligation adapter sequence of the first terminal oligonucleotide and the first orthogonal ligation adapter sequence of the targeting oligonucleotide, thereby bringing the first end of the first terminal oligonucleotide and the first end of the targeting oligonucleotide into close proximity; and
permitting the second end splint oligonucleotide to hybridize to the first orthogonal ligation adapter sequence of the second terminal oligonucleotide and the second orthogonal ligation adapter sequence of a signal oligonucleotide, thereby bringing the first end of the second terminal oligo and the second end of the signal oligonucleotide into close proximity; and
the ligating step further comprises:
ligating the first end of the first terminal oligonucleotide to the first end of the targeting oligonucleotide; and
ligating the first end of the second terminal oligonucleotide to the second end of the signal oligonucleotide.

40. The method of claim 37, further comprising amplifying the ligated probe precursor with primers that bind to the first primer biding site and the second primer binding site to provide an amplified probe precursor, transcribing the amplified probe precursor to provide an RNA probe precursor, and reverse transcribing the RNA probe precursor to provide a single stranded DNA probe construct.

41-42. (canceled)

43. The method of claim 41, further comprising:
(i) subjecting the amplified probe precursor to exonuclease digestion to provide a single stranded DNA probe construct;
(ii) subjecting the amplified probe precursor to DNA nicking and gel purification to provide a single stranded DNA probe construct; or
(iii) performing circle-to-circle DNA amplification to provide a single stranded DNA probe construct.

44-54. (canceled)
